(19) United States
(12) Patent Application Publication
(10) Pub. No.: US 2004/0241797 A1
(43) Pub. Date: Dec. 2, 2004

(54) Use of alphacp1, alphacp2, and hur for modulating gene expression and inducing angiogenesis

(76) Inventor: Louis-Georges Guy, Montreal (CA)

Correspondence Address: F Michael Sajovec, Myers Bigel Sibley & Sajovec, Suite 600, 4140 Parklake Avenue, Raleigh, NC, 27612, US

(21) Appl. No.: 10/486,865

(22) PCT Filed: Aug. 16, 2002
(86) PCT No.: PCT/CA02/01275

Related U.S. Application Data
(60) Provisional application No. 60/312,397, filed on Aug. 16, 2001.

(51) Int. Cl. C12P 21/02; C07K 14/475; C07H 21/04

(52) U.S. Cl. 435/69.1; 435/455; 435/320.1; 435/325; 530/399; 536/23.5

(57) ABSTRACT

The present invention describes methods for modulating gene expression, stabilizing VEGF mRNAs, for inducing angiogenesis, for treating various mammalian diseases including coronary and cardiac diseases and for identifying modulators of gene expression by using human proteins called αCP1, αCP2 and HuR. The invention also describes mRNA stabilizing elements and consensus sequences involved in binding of αCP1, αCP2 and HuR proteins to mRNAs.

### RELATED APPLICATIONS

This application claims priority of U.S. Provisional Application 60/312,397 filed Aug. 16, 2001, the disclosure of which is incorporated by reference herein in its entirety.

### BACKGROUND OF THE INVENTION

a) Field of the Invention

The present invention is concerned with the use of human proteins called αCP1, αCP2 and HuR for modulating gene expression, stabilizing VEGF mRNAs, for inducing angiogenesis, for treating various mammalian diseases including coronary and cardiac diseases and for identifying modulators of gene expression. The invention also relates to mRNA stabilizing elements and sequences involved in binding of αCP1, αCP2 and HuR proteins to mRNA.

b) Brief Description of the Prior Art

Chronic ischemic heart disease is a worldwide health problem of major proportions. According to the American Heart Association, 60.8 millions of Americans have at least one type of cardiovascular disease. In particular, 12.4 millions of Americans suffer from coronary heart disease (CHD) and almost 450 000 deaths in the United States alone were deemed to CHD. Current treatments include medication, percutaneous transluminal coronary angioplasty and coronary artery bypass surgery. These procedures are quite successful to increase blood flow in the myocardium and thus ameliorating the condition of the patient. However, due to the progressive nature of CHD, the beneficial effects of these procedures are not permanent and new obstructions can occur. Patients that live longer through effective cardiovascular interventions may eventually use up their treatment options. Also, an important patient population is still refractory to these treatments due to diffuse artherosclerotic disease and/or small caliber arteries.

A new field of treatment is therapeutic angiogenesis, which could be beneficial to this patient population. Angiogenesis is defined as blood vessel sprouting and proliferation from pre-existing vasculature. The net result is higher capillary density, and better blood perfusion. Many growth factors are currently used to induce angiogenesis (VEGF, FGF) but none of these factors has the property to stimulate each step of angiogenesis (basal membrane disruption, endothelial cell proliferation, migration and differentiation followed by periendothelial cells recruitment). Cell hypoxia can naturally induce a strong angiogenesis by triggering gene activation cascades that result in the synthesis and release of factors that will modulate each of the steps of angiogenesis. Regulators of hypoxia could be used to stimulate the synthesis of angiogenic factor. Many factors could be activated at once, resulting in an angiogenesis significantly stronger than with individual factors.

Regulators of hypoxia include the transcription factors of the Hypoxia Inducible Factors family (HIF-1α, HIF-2α and the newly discovered HIF-3α). In response to low oxygen tension, these factors are stabilized; and induce the transcription of hypoxia-inducible genes that help cell survival in hypoxia. These target genes are implicated in processes such as anaerobic metabolism (glucose transporters and glycolytic enzymes), vasodilatation (inducible nitric oxide synthase (iNOS) and heme oxygenase-1 (HO-1)), increased breathing (tyrosine hydroxylase (TH-1)), erythropoiesis (erythropoietin (EPO)) and angiogenesis (Vascular Endothelial Growth Factor (VEGF)). However, hypoxia gene induction can also occur post-transcriptionally through the modulation of mRNA stability. Transcripts of genes such as EPO, VEGF and TH-1 are labile and quickly degraded in normoxic conditions. However, in hypoxia, their degradation rate is slowed, resulting in longer half-life, and higher global steady-state levels of messenger RNAs.

VEGF mRNA is a labile messenger that is stabilized in hypoxia. That stabilization occurs through a discrete element in the 3′ untranslated region of the messenger. The protein HuR binds to that element as a complex with unknown proteins, in a hypoxia-specific fashion. Since HuR is present in normoxia as well as in hypoxia, these unknown proteins might thus be responsible for the hypoxia regulation of VEGF mRNA stabilization. As such, the genes encoding those proteins could be useful as angiogenic agents.

The gene encoding αCP1 has been known since 1994. It was cloned by several groups and often given different names: αCP1, hnRNP-E1, PCBP1 and nucleic acid binding protein sub2.3. αCP1 protein is usually described as a heterodimer with αCP2, a closely related protein. The literature about αCP1 indicates that it plays a role in the stabilization of four mRNAs: α-globin (a highly stable mRNA in differentiated erythroid cells), androgen receptor and two mRNAs stabilized by hypoxia: TH-1, and EPO. Furthermore, αCP1 was shown to bind 15-lipoxygenase mRNA and prevent its translation in the early stages of erythropoiesis.

Although the scientific advancements provided by the discoveries pertaining to HuR, αCP1 and αCP2, many problems remained unresolved. For instance, before the present invention, it was unknown that αCPs and HuR could bind each other. It was also unknown that αCP1 and αCP2 could bind and stabilize VEGF mRNA. Nucleic acid sequences onto which αCPs and HuR bind remained to be determined. Some isoforms of αCP1 and of αCP2 were also unknown.

There is therefore a need for novel isoforms of αCP that are capable of binding mRNAs molecules, such as VEGF mRNA, for stabilizing the same.

Furthermore, it would be highly desirable to be provided with methods for modulating gene expression turning to profit the fact that αCPs and HuR can bind each other. More particularly, it would be highly desirable to have methods for stabilizing VEGF mRNA and methods for inducing angiogenesis.

It would also be desirable to be provided with screening methods for identifying or selecting compounds capable of reducing expression of genes and more particularly Vascular Endothelial Growth Factor (VEGF), α-globin, Erythropoietin (EPO), 15-Lipoxygenase (15-LOX), and Androgen receptor (AR).

There is a further need for novel αCPs sequences and for nucleic acid sequences on which αCPs and HuR may bind.

There is also a long felt need for treatment methods wherein genes encoding the Vascular Endothelial Growth Factor (VEGF), α-globin, Erythropoietin (EPO), 15-Lipoxygenase (15-LOX), and Androgen receptor (AR) are involved, more particularly cancer, erythropoiesis, and diseases involving reduced levels of L-DOPA.

The present invention fulfils these needs and also other needs as it will be apparent to those skilled in the art upon reading the following specification.

### SUMMARY OF THE INVENTION

The present inventor has discovered novel human αCP isoforms (referred to hereinafter as αCP1A, αCP1B, αCP2A, and αCP2B).

The present inventor has also discovered that αCP polypeptides (αCPs) and HuR bind each other.

The present inventor has further discovered that αCP polypeptides are capable of binding mRNAs molecules, such as VEGF mRNA, for stabilizing the same. The present inventor has further discovered that HuR is capable of binding mRNA molecules previously unknown to be bound by HuR. Nucleic acid sequences on which αCPs and/or HuR binds have also been identified.

In general, the invention features an isolated or purified nucleic acid molecule, such as genomic, cDNA, antisense, DNA, RNA or a synthetic nucleic acid molecule that encodes or corresponds to a human αCP.

According to a first aspect, the invention features isolated or purified αCP nucleic acid molecules, human αCP polypeptides and fragment thereof. The invention also features substantially pure human polypeptides and proteins that are encoded by any of the above mentioned nucleic acids. In one embodiment, the protein has the biological activity of a human αCP polypeptide.

According to another aspect, the invention features a purified antibody. In a preferred embodiment, the antibody is a monoclonal or a polyclonal antibody that specifically binds to a purified mammalian αCP polypeptide.

In another aspect, the present invention further features a method for modulating gene expression in a cell, comprising modulating a binding interaction between a αCP polypeptide and a HuR polypeptide.

In a more specific aspect, the present invention further features methods for modulating VEGF expression in a mammalian cell, the method comprising the step of modulating in the cell a binding interaction between a αCP polypeptide and a HuR polypeptide. In a preferred embodiment, the method is carried out for increasing VEGF expression.

In another related aspect, the invention provides a method for modulating VEGF expression in a mammalian cell, the method comprising the step of modulating in the cell a binding interaction between a αCP polypeptide and a VEGF mRNA.

In another related aspect, the invention provides method for modulating VEGF mRNA stability or resistance to degradation in a mammalian cell, the method comprising the step of modulating in the cell a binding interaction between a αCP polypeptide and the VEGF mRNA.

Furthermore, the present invention features methods for inducing angiogenesis in a mammalian tissue having a plurality of cells. In one embodiment the method comprises the step of permitting or increasing in cells of the tissue a binding interaction between a αCP polypeptide and a VEGF mRNA; and/or between a αCP polypeptide and a HuR polypeptide.

The present invention also features a method for modulating gene expression in a cell, comprising modulating binding of αCP polypeptide to a mRNA stabilizing element of a nucleic acid molecule that is present into the cell, the mRNA stabilizing element comprising SEQ ID NO:19, SEQ ID NO:20 and/or SEQ ID NO: 21.

The present invention further features an isolated or purified nucleic acid molecule comprising a mRNA stabilizing element, the mRNA stabilizing element comprising a nucleic acid sequence selected from the group consisting of: SEQ ID NO:19, SEQ ID NO:20, SEQ ID NO:21, SEQ ID NO:22, SEQ ID NO:23, and complementary sequences thereof. The invention also encompasses expression vectors comprising such mRNA stabilizing element(s) and methods of using the same for expressing a gene product under conditions of hypoxia.

The invention further provides method for selecting a compound that is capable of reducing gene expression and more particularly compounds that are capable of reducing VEGF expression.

The present invention further features a method for modulating tumoral cell survival or for eliminating a tumoral cell in a mammal, a method for reducing anemia in a mammal, and a method for treating a mammalian disease involving reduced levels of L-DOPA.

One of the greatest advantages of the present invention is that it provides nucleic acid molecules, proteins, polypeptides, antibodies, probes, and cells that can be used for modulating gene expression and more particularly promote angiogenesis.

Other objects and advantages of the present invention will be apparent upon reading the following non-restrictive description of the preferred embodiments thereof and from the claims.

### BRIEF DESCRIPTION OF THE DRAWINGS

FIG. 1 is a picture of a Northern blot showing VEGF induction in hypoxia.

FIGS. 2A and 2B are pictures of SDS-PAGE analysis showing in vitro interaction between αCP1 and HuR.

FIG. 3 is a bar graph illustrating VEGF expression after αCP1 transfection of 5 independent HEK 293 cell populations.

FIG. 4 is a sequence alignment of human VEGF mRNA 3′-Untranslated regions (3′UTRs) with known Hypoxia-Inducible Protein-Binding Sequence (HIPBS) or other αCP binding sites. A potential αCP binding site consensus sequence is also shown. Y: Pyrimidine (C or U); N: Any nucleotide. Numbering of the sequences starts at the beginning of the 3′UTRs.

FIG. 5 is a sequence alignment of human VEGF, human Androgen receptor (AR) and human 15-Lipoxygenase (h15-LOX) 3′UTRs and p21. A consensus sequence is also shown. Numbering of the sequences starts at the beginning of the 3′UTRs.

### DETAILED DESCRIPTION OF THE INVENTION

### A) Definitions

Throughout the text, the word “kilobase” is generally abbreviated as “kb”, the words “deoxyribonucleic acid” as “DNA”, the words “ribonucleic acid” as “RNA”, the words “messenger ribonucleic acid” as “mRNA”, the words “complementary DNA” as “cDNA”, the words “polymerase chain reaction” as “PCR”, and the words “reverse transcription” as “RT”. Nucleotide sequences are written in the 5′ to 3′ orientation unless stated otherwise.

In order to provide an even clearer and more consistent understanding of the specification and the claims, including the scope given herein to such terms, the following definitions are provided:

αCP nucleic acid: means any nucleic acid (see hereinafter) encoding a mammalian polypeptide that has the biological activity of binding mRNAs and thereby stabilizes and/or increases the mRNA's resistance to degradation and having at least 75%, 77%, 80%, 85%, 90%, 95%, 97% or 100% identity or similarity to: i) the nucleic acids set forth SEQ. ID. NOs: 1, 3, 5, 7, or ii) other αCP nucleic acids known in the art (see hereinafter). αCP protein or αCP polypeptide: means a protein, a polypeptide, or a fragment thereof, encoded by a αCP nucleic acid as described above. Human αCP polypeptides having at least 75%, 77%, 80%, 85%, 90%, 95%, 97% or 100% identity or similarity to: i) the amino acid sequences set forth SEQ. ID. NOs: 2, 4, 6, 8; or ii) other αCP polypeptides known in the art (see hereinafter) are more particularly concerned.

Antisense: Refers to nucleic acid molecules capable of regulating the expression of a corresponding gene in humans and animals. An antisense molecule as used herein may also encompass a gene construct comprising a structural genomic gene, a cDNA gene or part thereof in reverse orientation relative to its or another promoter. Typically antisense nucleic acid sequences are not templates for protein synthesis but yet interact with complementary sequences in other molecules (such as a gene or RNA), thereby causing the function of those molecules to be affected.

Binding interaction/Binding affinity: Refers to quality, state or process of the attraction or adherence of two molecules to one another. Typically, binding occurs because the shape and chemical natures of parts of the molecule surfaces are complementary and/or have a relatively high attraction or affinity for each other. As used herein, it generally refers to the binding of a αCP polypeptide with a HuR polypeptide, or the binding of a αCP polypeptide or a HuR polypeptide to a nucleic acid molecule (e.g. mRNA).

Expression: refers to the process by which gene encoded information is converted into the structures present and operating in the cell. In the case of cDNAs, cDNA fragments and genomic DNA fragments, the transcribed nucleic acid is subsequently translated into a peptide or a protein in order to carry out its function if any. By “positioned for expression” is meant that the DNA molecule is positioned adjacent to a DNA sequence which directs transcription and translation of the sequence (i.e., facilitates the production of, e.g., a αCP polypeptide, a recombinant protein or a RNA molecule).

Fragment: Refers to a section of a molecule, such as a protein, a polypeptide or a nucleic acid, and is meant to refer to any portion of the amino acid or nucleotide sequence.

Homolog: refers to a nucleic acid molecule or polypeptide that shares similarities in DNA or protein sequences.

Host: A cell, tissue, organ or organism capable of providing cellular components for allowing the expression of an exogenous nucleic acid embedded into,a vector or a viral genome, and for allowing the production of viral particles encoded by such vector or viral genome. This term is intended to also include hosts which have been modified in order to accomplish these functions. Bacteria, fungi, animal (cells, tissues, or organisms) and plant (cells, tissues, or organisms) are examples of a host.

HuR nucleic acid: means any nucleic acid (see hereinafter) encoding a mammalian polypeptide that has the biological activity of binding mRNAs and/or inhibiting specific endonucleases that degrade mRNA, thereby stabilizing and/or increasing the mRNA's resistance to degradation, and having at least 75%, 77%, 80%, 85%, 90%, 95%, 97% or 100% identity or similarity to human HuR nucleic acids known in the art (see hereinafter). HuR protein or HuR polypeptide: means a protein, a polypeptide, or a fragment thereof, encoded by a HuR nucleic acid as described above. Human HuR polypeptides having at least 75%, 77%, 80%, 85%, 90%, 95%, 97% or 100% identity or similarity to human HuR polypeptide known in the art (see hereinafter) are more particularly concerned.

Isolated or Purified or Substantially pure: Means altered “by the hand of man” from its natural state, i.e., if it occurs in nature, it has been changed or removed from its original environment, or both. For example, a polynucleotide or a protein/peptide naturally present in a living organism is not “isolated”, the same polynucleotide separated from the coexisting materials of its natural state, obtained by cloning, amplification and/or chemical synthesis is “isolated” as the term is employed herein. Moreover, a polynucleotide or a protein/peptide that is introduced into an organism by transformation, genetic manipulation or by any other recombinant method is “isolated” even if it is still present in said organism.

Modulation: Refers to the process by which a given variable is regulated to a certain proportion.

Nucleic acid: Any DNA, RNA sequence or molecule having one nucleotide or more, including nucleotide sequences encoding a complete gene. The term is intended to encompass all nucleic acids whether occurring naturally or non-naturally in a particular cell, tissue or organism. This includes DNA and fragments thereof, RNA and fragments thereof, cDNAs and fragments thereof, expressed sequence tags, artificial sequences including randomized artificial sequences.

Polypeptide: means any chain of more than two amino acids, regardless of post-translational modification such as glycosylation or phosphorylation.

Percent identity and Percent similarity: used herein in comparisons of nucleic acid and/or among amino acid sequences. Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Owl 53705). This software program matches similar sequences by assigning degrees of similarity to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

Ribozymes: means a RNA molecule with enzymatic property against a specific RNA target, resulting in the degradation of this RNA.

Specifically binds: means a compound that recognizes and binds a protein or polypeptide but that does not substantially recognizes and bind other molecules in a sample, e.g., a biological sample, that naturally includes protein. The compound may consists but is not limited to a protein, a peptide, an antibody, a chemical, etc.

Substantially pure polypeptide: means a polypeptide that has been separated from the components that naturally accompany it. Typically, the polypeptide is substantially pure when it is at least 60%, by weight, free from the proteins and naturally-occurring organic molecules with which it is naturally associated. Preferably, the polypeptide is a αCP polypeptide that is at least 75%, 80%, or 85%, more preferably at least 90%, 95% or 97% and most preferably at least 99%, by weight, pure. A substantially pure αCP polypeptide may be obtained, for example, by extraction from a natural source (including but not limited to lung cells, kidney cells, heart cells or any other cell expressing αCP) by expression of a recombinant nucleic acid encoding a αCP polypeptide in an expression system such as E Coli or Bacculovirus, or by chemically synthesizing the protein. Purity can be measured by any appropriate method, e.g., by column chromatography, polyacrylamide gel electrophoresis, or HPLC analysis. A protein is substantially free from naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes. By “substantially pure DNA” is meant DNA that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA which is part of a hybrid gene encoding an additional polypeptide sequence.

Transformed or Transfected or Transduced or Transgenic cell: refers to a cell into which (or into an ancestor of which) has been introduced, by means of recombinant DNA techniques, a DNA molecule encoding (as used herein) a αCP polypeptide. By “transformation” is meant any method for introducing foreign molecules into a cell. Lipofection, calcium phosphate precipitation, retroviral delivery, electroporation, and ballistic transformation are just a few of the teachings which may be used.

Transgenic animal: any animal having a cell which includes a DNA sequence which has been inserted by artifice into the cell and becomes part of the genome of the animal which develops from that cell. As used herein, the transgenic animals are usually mammalian (e.g., rodents such as rats or mice) and the DNA (transgene) is inserted by artifice into the nuclear genome.

Vector: A self-replicating RNA or DNA molecule which can be used to transfer an RNA or DNA segment from one organism to another. Vectors are particularly useful for manipulating genetic constructs and different vectors may have properties particularly appropriate to express protein(s) in a recipient during cloning procedures and may comprise different selectable markers. Bacterial plasmids are commonly used vectors. Modified viruses such as adenoviruses and retroviruses are other examples of vectors.

### B) General Overview of the Invention

The invention generally concerns αCP polypeptides (αCPs) and HuR. The present inventor has discovered novel isoforms of human αCPs. The present inventor has also discovered that αCP polypeptides and HuR bind each other. The present inventor has further discovered that αCP polypeptides are capable of binding mRNAs molecules, such as VEGF mRNA, for stabilizing the same. The nucleic acid sequences on which αCPs and/or HuR binds have also being identified. Accordingly, expression of gene(s) in a mammalian cell may be modulated at the mRNA level and leads to numerous applications in various areas such as cardiovascular diseases, cancer, anemia, and neuronal disorders. For instance, VEGF expression may be increased for inducing angiogenesis in mammalian tissues.

The invention also concerns screening methods for identifying or selecting compounds capable of modulating gene expression at the mRNA level.

### i) Cloning and Molecular Characterization of αCPs

As it will be described hereinafter in the exemplification section of the invention, the inventors have discovered, cloned and sequenced many isoforms of human αCP (referred hereinafter as αCP1A, αCP1B, αCP2A and αCP2B).

According to the known sequence of the αCP genes (GenBank™ No. U24223 and X78136), DNA oligonucleotides were designed to amplify the complete sequence by reverse transcription of human mRNA followed by PCR reactions, cloned in a plasmid vector and sequenced. Two isoforms of αCP1 were obtained and were labeled αCP1A and αCP1B. The sequence of the αCP1A cDNA and predicted amino acid sequence is shown in the “Sequence Listing” section. SEQ ID NO: 1 corresponds to the human αCP1A cDNA and SEQ ID NO: 2 corresponds to the predicted amino acid sequence of the human protein. αCP1B cDNA and predicted amino acid sequence correspond to SEQ ID NO: 3 and SEQ ID NO: 4, respectively.

αCP1A is almost identical to the previously known sequence (99% amino acid identity to GenBank™ No. U24223) with only 2 amino acid substitutions (amino acid 135 G to U and amino acid 306 R to C). αCP1B is missing a protein domain (amino acids 37 to 47, corresponding to nucleotides 286-318 of GenBank™ No. U24223) and is probably generated by alternative splicing, it is 100% identical to U24223 for the conserved exons and the deletion of the spliced out exon reduces identity to 96%.

αCP2 was also amplified and 2 cloned sequences (αCP2A and αCP2B) represent two isoforms of that factor. αCP2A cDNA and predicted amino acid sequence correspond to SEQ ID NO: 5 and SEQ ID NO: 6, whereas αCP2B cDNA and predicted amino acid sequence correspond to SEQ ID NO: 7 and SEQ ID NO: 8 respectively. αCP2A has got 2 missing domains (amino acids 169-172 and 269-279 or nucleotides 525-536 and 821-857) compared to known sequence (GenBank™ No. X78136, that we renamed αCP2C). αCP2B also misses 2 domains (amino acids 198-228 and 269-279 or nucleotides 612-704 and 822-857) compared to αCP2C. Identity of the cloned sequences to GenBank™ No. X78136 is 100% in the conserved exons, but splicing reduces it to 95% for αCP2A and 87% for αCP2B.

In view of the above, it seems that αCP1A, αCP1B, αCP2A and αCP2B nucleic acids and amino acids sequences are novel. Therefore, the present invention concerns an isolated or purified nucleic acid molecule (such as cDNA) comprising a sequence selected from the group consisting of:

a) a sequence as set forth in SEQ ID NO: 1 or 3;

b) a sequence encoding an amino acid sequence as set forth in SEQ ID NO: 2 or 4;

c) a sequence as set forth in SEQ ID NO: 5 or 7; and

d) a sequence encoding an amino acid sequence as set forth in SEQ ID NO: 6 or 8.

In a related aspect, the present invention concerns an isolated or purified polypeptide, comprising an amino acid sequence selected from the group consisting of:

a) sequences encoded by a nucleic acid of claim 1;

b) sequences comprising an amino acid sequence as set forth in SEQ ID NO: 2, SEQ ID NO: 4, SEQ ID NO: 6 or SEQ ID NO:8.

### ii) Vectors, Cells and Transgenic Animals

The invention is also directed to a host, such as a genetically modified cell, expressing a functional human αCP1A, αCP1B, αCP2A and/or αCP2B polypeptide as defined previously. Preferably, the cell is a skeletal muscular cell or a cardiac cell.

The αCP1A, αCP1B, αCP2A and/or αCP2B expressing cell may be a transiently-transfected mammalian cell line (such as HEK293 cells, a Hep3B cells, and the like) or any suitable isolated primary cells, including by not limited to mammalian skeletal muscular cells, cardiac cells, bone marrow cells, fibroblasts, smooth muscle cells, endothelial cells, endothelial progenitor cells and embryonic stem cells

A number of vectors suitable for stable transfection of mammalian cells are available to the public (e.g. plasmids, adenoviruses, adeno-associated viruses, retroviruses, Herpes Simplex Viruses, Alphaviruses, Lentiviruses), as are methods for constructing such cell lines. The present invention encompasses any type of vector with a αCP1A, αCP1B, αCP2A and/or αCP2B sequence.

The cells of the invention may be particularly useful when transplanted in a compatible recipient for inducing angiogenesis, relieving ischemia, increasing the metabolic activity of a mammalian muscular tissue, and/or increasing muscular function in CHF or in peripheral vascular disease, locally or in surrounding transplanted tissue. Of course, the genetically modified cells of the present invention could also be used for the formation of artificial organs or for tissue constructions. αCP1A, αCP1B, αCP2A and/or αCP2B expressing cells may also be used for producing αCP1A, αCP1B, αCP2A and/or αCP2B and derivatives thereof (see hereinafter).

The mammalian αCP1A, αCP1B, αCP2A and/or αCP2B according to the present invention or a fragment thereof may also be used to generate 1) transgenic animals that express the αCP gene(s) or αCP mutants at various levels in one or multiple cell lineages, 2) knock-out animal in which expression of the endogenous αCP gene(s) is either prevented or regulated in one or multiple cell lineages.

Characterization of αCP genes provides information that is necessary for a αCP1A, αCP1B, αCP2A and/or αCP2B knockout animal model to be developed by homologous recombination. Preferably, the model is a mammalian animal, most preferably a mouse. Similarly, an animal model of αCP1A, αCP1B, αCP2A and/or αCP2B overproduction may be generated by integrating one or more αCP sequences into the genome, according to standard transgenic techniques.

### iii) Synthesis of αCP1A and/or αCP1B and Functional Derivative Thereof

Knowledge of human αCP1A, αCP1B, αCP2A and/or αCP2B sequences open the door to a series of applications. For instance, the characteristics of the cloned αCP1A, αCP1B, αCP2A and/or αCP2B sequences may be analyzed by introducing the sequence into various cell types or using in vitro extracellular systems. The function of αCP1A, αCP1B, αCP2A and/or αCP2B may then be examined under different physiological conditions. The αCP cDNA sequences may be manipulated in studies to understand the expression of the gene and gene product. Alternatively, cell lines may be produced which overexpress the gene product allowing purification of αCP1A, αCP1B, αCP2A and/or αCP2B for biochemical characterization, large-scale production, antibody production, and patient therapy.

For protein expression, eukaryotic and prokaryotic expression systems may be generated in which the αCP sequences are introduced into a plasmid or other vector which is then introduced into living cells. Constructs or expression plasmid in which the αCP cDNA sequences containing the entire open reading frame is inserted in the correct orientation may be used for protein expression. Alternatively, portions of the sequence, including wild-type or mutant αCP sequences, may be inserted. Prokaryotic and eukaryotic expression systems allow various important functional domains of the protein to be recovered as fusion proteins and then used for binding, structural and functional studies and also for the generation of appropriate antibodies.

Eukaryotic expression systems permit appropriate post-translational modifications of expressed proteins. This allows for studies of the αCP gene and gene product including determination of proper expression and post-translational modifications for biological activity, identifying regulatory elements located in the 5′ region of the αCP gene and their role in tissue regulation of protein expression. It also permits the production of large amounts of normal and mutant proteins for isolation and purification, to use cells expressing αCP1A, αCP1B, αCP2A and/or αCP2B as a functional assay system for antibodies generated against the protein, to test the effectiveness of pharmacological agents or as a component of a signal transduction system, to study the function of the normal complete protein, specific portions of the protein, or of naturally occurring polymorphisms and artificially produced mutated proteins. The αCP DNA sequences may be altered by using procedures such as restriction enzyme digestion, DNA polymerase fill-in, exonuclease deletion, terminal deoxynucleotide transferase extension, ligation of synthetic or cloned DNA sequences and site directed sequence alteration using specific oligonucleotides together with PCR.

Accordingly, the invention also concerns a method for producing a human a human αCP1A, αCP1B, αCP2A and/or αCP2B polypeptides. The method comprises the steps of: (i) providing a cell transformed with a nucleic acid sequence encoding a human αCP1A, αCP1B, αCP2A and/or αCP2B polypeptide positioned for expression in the cell; (ii) culturing the transformed cell under conditions suitable for expressing the nucleic acid; (iii) producing said a human αCP1A, αCP1B, αCP2A and/or αCP2B polypeptides; and optionally, (iv) recovering the human αCP polypeptides produced.

Once the recombinant protein is expressed, it is isolated by, for example, affinity chromatography. In one example, an anti-αCP1A, anti-αCP1B, anti-αCP2A and/or anti-αCP2B antibodies, which may be produced by the methods described herein, can be attached to a column and used to isolate the αCP proteins. Lysis and fractionation of αCP-harboring cells prior to affinity chromatography may be performed by standard methods. Once isolated, the recombinant protein can, if desired, be purified further.

Methods and techniques for expressing recombinant proteins and foreign sequences in prokaryotes and eukaryotes are well known in the art and will not be described in more detail. One can refer, if necessary to Joseph Sambrook, David W. Russell, Joe Sambrook Molecular Cloning: A Laboratory Manual 2001 Cold Spring Harbor Laboratory Press. Those skilled in the art of molecular biology will understand that a wide variety of expression systems may be used to produce the recombinant protein. The precise host cell used is not critical to the invention.

Polypeptides of the invention, particularly short αCP1A, αCP1B, αCP2A and/or αCP2B fragments, may also be produced by chemical synthesis. These general techniques of polypeptide expression and purification can also be used to produce and isolate-useful αCP fragments or analogs, as described herein.

Skilled artisans will recognize that a mammalian αCP1A, αCP1B, αCP2A and/or αCP2B, or a fragment thereof (as described herein), may serve as an active ingredient in a therapeutic composition. This composition, depending on the αCP or fragment included, may be used to regulate cell proliferation, survival, metabolism and angiogenesis and thereby treat any condition that is caused by a disturbance in cell proliferation, accumulation, or metabolism. Thus, it will be understood that another aspect of the invention described herein, includes the compounds of the invention in a pharmaceutically acceptable carrier.

### iv) αCP1A, αCP1B, αCP2A and/or αCP2B Antibodies

The invention features purified antibodies that specifically binds to a αCP1A, αCP1B, αCP2A and/or αCP2B polypeptide. The antibodies of the invention may be prepared by a variety of methods using the αCP1A, αCP1B, αCP2A and/or αCP2B polypeptides described above. For example, the αCP polypeptides, or antigenic fragments thereof, may be administered to an animal in order to induce the production of polyclonal antibodies. Alternatively, antibodies used as described herein may be monoclonal antibodies, which are prepared using hybridoma technology (see, e.g., Hammerling et al., In Monoclonal Antibodies and T-Cell Hybridomas, Elsevier, N.Y., 1981). The invention features antibodies that specifically bind human αCP1A, αCP1B, αCP2A and/or αCP2B polypeptides, or fragments thereof. In particular, the invention features “neutralizing” antibodies. By “neutralizing” antibodies is meant antibodies that interfere with any of the biological activities of the αCP1A, αCP1B, αCP2A and/or αCP2B polypeptide, particularly the ability of αCP to bind HuR. The neutralizing antibody may reduce the ability of αCP polypeptides to bind HuR by, preferably 50%, more preferably by 70%, and most preferably by 90% or more. The neutralizing antibody can also interfere with RNA binding activity of αCP and/or HuR. Any standard assay, including those described herein, may be used to assess potentially neutralizing antibodies. Once produced, monoclonal and polyclonal antibodies are preferably tested for specific αCP1A, αCP1B, αCP2A and/or αCP2B recognition by Western blot, immunoprecipitation analysis or any other suitable method.

In addition to intact monoclonal and polyclonal anti-αCP antibodies, the invention features various genetically engineered antibodies, humanized antibodies, and antibody fragments, including F(ab′)2, Fab′, Fab, Fv and sFv fragments. Antibodies can be humanized by methods known in the art. Fully human antibodies, such as those expressed in transgenic animals, are also features of the invention.

Antibodies that specifically recognize αCP1A, αCP1B, αCP2A and/or αCP2B (or fragments thereof), such as those described herein, are considered useful to the invention. Such an antibody may be used in any standard immunodetection method for the detection, quantification, and purification of αCP. The antibody may be a monoclonal or a polyclonal antibody and may be modified for diagnostic or for therapeutic purposes.

The antibodies of the invention may, for example, be used in an immunoassay to monitor αCP1A, αCP1B, αCP2A and/or αCP2B expression levels, to determine the subcellular location of αCPs or to determine the amount of αCPs or a fragment thereof in a biological sample. Antibodies that inhibit αCP1A, αCP1B, αCP2A and/or αCP2B described herein may be especially useful for conditions where decreased αCPs function would be advantageous such as inhibition of cancer cell proliferation (see hereinafter). In addition, the antibodies may be coupled to compounds for diagnostic and/or therapeutic uses such as radionucleotides for imaging and therapy and liposomes for the targeting of compounds to a specific tissue location. The antibodies may also be labeled (e.g. immunofluorescence) for easier detection.

### v) Assessment of αCP1A, αCP1B, αCP2A and/or αCP2B Intracellular or Extracellular Levels

As noted, the antibodies and probes described above may be used to monitor αCP1A, αCP1B, αCP2A and/or αCP2B protein expression and/or to determine the amount of αCPs in a biological sample.

In addition, in situ hybridization may be used to detect the expression of the αCP1A, αCP1B, αCP2A and/or αCP2B isoforms. As it is well known in the art, in situ hybridization relies upon the hybridization of a specifically labeled nucleic acid probe to the cellular RNA in individual cells or tissues. Therefore, oligonucleotides or cloned nucleotide (RNA or DNA) fragments corresponding to unique portions of the αCP1A, αCP1B, αCP2A and/or αCP2B isoforms may be used to assess αCPs cellular levels or detect specific mRNA species. Such an assessment may also be done in vitro using well known methods (Northern analysis, quantitative PCR, etc.).

Determination of the amount of αCPs or fragment thereof in a biological sample may be especially useful for diagnosing a cell proliferation disease or an increased likelihood of such a disease, particularly in a human subject, using a αCP nucleic acid probe or antibody. The present inventor also suspects that a correlation exists between tumor presence and/or progression and the amount αCPs.

The methods of the invention may be carried out by contacting, in vitro or in vivo; a biological sample (such as a blood sample or a tissue biopsy) from an individual suspected of having cancer, with a αCP antibody or a probe according to the invention, in order to evaluate the amount of αCP1A, αCP1B, αCP2A and/or αCP2B in the sample or the cells therein. The measured amount would be indicative of the probability of the subject of tumor presence and/or progression since it is expected that increased level of αCP1A and/or αCP1B expression can lead to higher levels of oncogenic factors such as VEGF and androgen receptor.

In a related aspect, the invention features a method for detecting the expression of αCP1A, αCP1B, αCP2A and/or αCP2B in tissues comprising, i) providing a tissue or cellular sample; ii) incubating said sample with an anti-αCP1A, anti-αCP1B anti-αCP2A and/or anti-αCP2B polyclonal or monoclonal antibody; and iii) visualizing the distribution of αCP.

Assay kits for determining the amount of αCP1A, αCP1B, αCP2A and/or αCP2B in a sample would also be useful and are within the scope of the present invention. Such a kit would preferably comprise αCP antibody(ies) or probe(s) according to the invention and at least one element selected from the group consisting of instructions for using the kit, assay tubes, enzymes, reagents or reaction buffer(s), enzyme(s).

### vi) αCP Binds to HuR

As mentioned previously, the present inventor has unexpectedly found that αCP polypeptides (αCPs) and HuR bind each other. Since both αCP and HuR are polypeptides known to bind mRNAs and participate in mRNAs stabilization, one aspect of the invention relates to methods for modulating gene expression in a cell, comprising modulating a binding interaction between a αCP polypeptide and a HuR polypeptide. According to the invention, such binding interaction increases stability of the mRNA(s), thereby increasing expression of the gene. Preferably, the gene consists of a gene which gives rise to mRNAs on which αCP polypeptide(s) and/or HuR polypeptide(s) binds. In one embodiment, expression of the gene is reduced by inhibiting or blocking the αCP-HuR binding interaction. In another embodiment, expression of the gene is increased by permitting or increasing the αCP-HuR binding interaction.

Another aspect of the invention relates to methods for modulating gene expression in a cell at the mRNA level, comprising modulating a binding interaction between a αCP polypeptide and/or a HuR polypeptide to mRNAs derived from the gene for which modulation is desired. Indeed, given that both αCP and HuR are polypeptides known to bind mRNAs and participate in mRNAs stabilization, and that αCP and HuR bind each other, it is highly probable that both αCP and HuR bind to some extent to the same mRNAs. This is demonstrated in the exemplification section for VEGF mRNA. Nucleic acid sequences that are binding sites for αCP and/or HuR binding is also given hereinafter.

According to the invention, the αCP polypeptide may consists of αCP1A, αCP1B, αCP2A, αCP2B and αCP2C (see hereinbefore). Of course ?other αCP1 isoforms or αCP2 isoforms yet to be discovered and having substantially the same biological activity than those described herein could also be used or targeted. The HuR polypeptide may consist of the known HuR polypeptides (GenBank™ XM_{—}166730) or other HuR isoforms yet to be discovered having substantially the same biological activity.

Although VEGF is a gene for which the present inventor has put much of its effort, and more particularly in increasing its expression for inducing angiogenesis, it is to be understood that the invention encompasses many others genes. A non limitative list of genes which could be modulated according to the methods of invention is given hereinafter.

One of the best studied role of αCP is stabilization of α-globin mRNA (X Wand et al. (1995) Mol Cell Biol 15: 1769-77; M Kiledjian et al. (1995) EMBO J. 14:4357-4364), one of the most stable (if not the most stable) mRNA. It is part of the complex α (hence the name αCP1 and αCP2). In view of the above, it is proposed that HuR is also part of the complex α and that it plays a role in α-globin mRNA stabilization. Modulation of the binding interaction between αCP and HuR would permit to modulate accordingly α-globin gene expression. The same holds true for the modulation of HuR binding to α-globin mRNA.

αCP1 and αCP2 binding to erythropoietin (EPO) mRNA has also been documented (M F Czyzik-Krzeska et A C Bendixen (1999) Blood 93:2111). Therefore, it is very likely that HuR participates in EPO mRNA stabilization also and that modulation of HuR binding to EPO mRNA and/or modulation of the binding interaction of between αCP and a HuR would permit to modulate accordingly EPO gene expression.

15-lipoxygenase (15-LOX) is known to be implicated in the formation of leukotrienes acting as mediators of inflammation and angiogenesis. 15-LOX is essential for erythroid cell differentiation. αCP1 was shown to bind 15-LOX mRNA and regulate its translation and stability (D H Ostareck et al. (1999) Cell 89: 597; M. Holcik et S A Liebhaber (1997) Proc Natl Acad Sci USA 94: 2410-14). It is therefore proposed that modulating HuR-αCP interaction could have an impact on inflammation. Also, leukotrienes being involved in angiogenesis, it is possible that modulation of HuR-αCP interaction could also have an effect there. The same holds true for the modulation of HuR binding to 15-LOX mRNA.

Tyrosine Hydroxylase (TH) is a rate-limiting enzyme in the synthesis of catecholamines, regulating breathing, many dystonia syndromes, Parkingson and seizures, among others. TH mRNA stability is regulate by αCP binding (M. Holcik et S A Liebhaber (1997) Proc Natl Acad Sci USA 94: 2410-14; W R Paulding et al. (1999) J Biol Chem 274: 2532-2538). Once again, HuR might also be implicated and modulation of HuR binding to TH mRNA and/or modulation of the αCP-HuR binding interaction could probably permit to modulate accordingly TH gene expression.

αCP binds also to the Androgen receptor (AR) mRNA. HuR also binds another region of the same mRNA in a cooperative manner with αCP (B B Yeap et al. (2002) J Biol Chem 277: 27183-27192). Modulation of the αCP-HuR binding interaction could probably permit to modulate accordingly AR gene expression in human prostate cancer, for example.

It is also known that HuR binds to AU-rich elements in the 3′ UTR of unstable mRNAs and stabilizes them. Many of those are proto-oncogenes having a short-lived mRNA including: GM-CSF (V E Myer et al. (1997) EMBO J. 16:2130-2139), VEGF (N S Levy et al. (1998) J Biol Chem 273: 6417-6423), c-fos (X C Fan et al. (1998) EMBO J. 17:3448-3460; W-J Ma et al. (1996) J Biol Chem 271: 8144-8151), p27^{KIP1} (S S Millard et al. (2000) Mol Cell Biol 20: 5947-5959), cyclin A (W Wang et al. (2000) EMBO J. 19:2340-2350), cyclin B1 (W Wang et al. (2000) EMBO J. 19:2340-2350), IL-3 (W-J Ma et al. (1996) J Biol Chem 271: 8144-8151), c-myc (W-J Ma et al. (1996) J Biol Chem 271: 8144-8151), p21^{WAF} (W Wang et al. (2000) Mol Cell Biol 20: 760-769), Neurofibromin (J Haeussler et al. (2000) Bioch. Biophys. Res. Comm. 267: 726-32), cyclooxygenase-2 (D A Dixon et al. (2001) J. Clin. Invest. 108: 1657-1665), androgen receptor (B B Yeap et al. (2002) J Biol Chem 277: 27183-27192), and iNOS (F Rodriguez-Pascual et al. (2000) J Biol Chem 275: 26040-26049). Proposed mechanisms of action of HuR include inhibition of specific endonuclease that degrades the unstable mRNA (Z Zhao et al. (2000) Nucleic Acids Res. 28: 2695-2701) and stabilization of the poly(A) through direct interaction (W-J Ma et al. (1997) Nucleic Acids Res. 25: 3564-3569). αCP might also be implicated and modulation of αCP binding to these mRNAs and/or modulation of the αCP-HuR binding interaction could probably permit to modulate expression of these proto-oncogenes and possibly reduce tumorigenicity accordingly.

### vii) mRNA Stabilizing Element and use Thereof for Modulating Gene Expression Regulated by αCP

As it will be demonstrated in the Exemplification section, the present inventor has found that αCP binds to VEGF mRNA 3′UTR region. Since it was already known that αCP bind other mRNAs, VEGF mRNA 3′UTR was analyzed for homologies with other human mRNA 3′UTR sequences known to be regulated by αCP factors (erythropoietin -(EPO; Genbank™ NM_{—}000799), α-Globin (aGlobin; Genbank™ V00489), Tyrosine Hydroxylase (TH; Genbank™ NM_{—}000360), 15-lipoxygenase (15LOX; Genbank™ M23892), androgen receptor (AR; Genbank™ M20132) and p21 (Genbank™ NM_{—}000389).

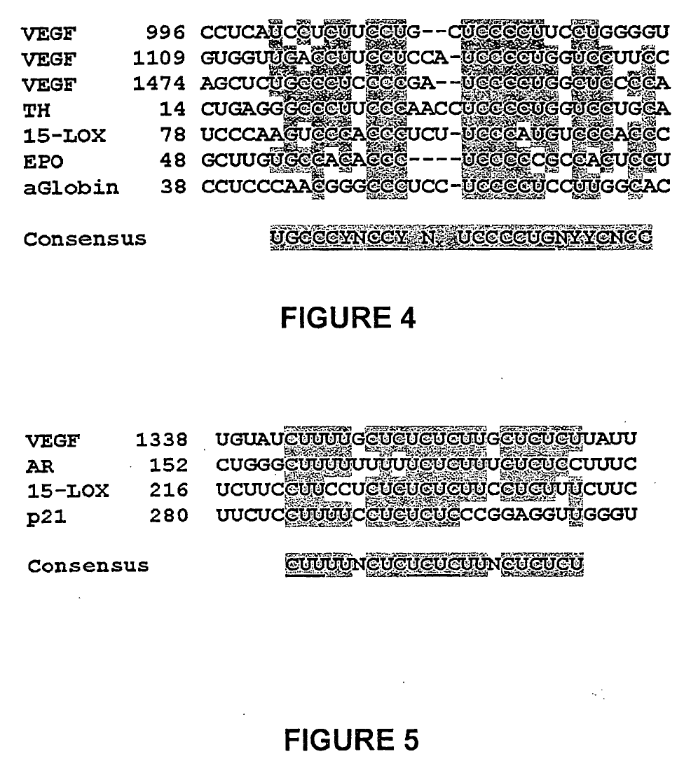

As shown in FIG. 4, an element with homology to sequences found in other αCP regulated mRNA was found in three regions in VEGF 3′UTR. The identified regions were previously described as Hypoxia-Inducible Protein-Binding-Site (HIPBS) of EPO and TH, αComplex binding site of α-globin and Differentiation Control Element (DICE) of 15-LOX. All those sequences are known or suspected to be αCP binding sites. In view of the above, it is assumed that αCP bind to these elements in VEGF mRNA 3′UTR as well and more particularly to regions 996-1027, 1109-1141 and/or 1474-1505. Therefore, another aspect of the invention concerns an isolated or purified nucleic acid molecule comprising a VEGF mRNA stabilizing element for αCP binding, this stabilizing element comprising a nucleic acid sequence selected from the group consisting of: SEQ ID NO:19, SEQ ID NO:20, SEQ ID NO:21, and complementary sequences thereof.

As shown also in FIG. 4, the sequences alignment also permitted to identify consensus sequences common for all these known αCP regulated genes. Underligned is the core of the consensus. Previous consensus sequence included only 6 nucleotides, namely YYCCCU (SEQ ID NO:24). Therefore, another aspect of the invention concerns an isolated or purified nucleic acid molecule comprising a mRNA stabilizing element for αCP binding, this stabilizing element comprising a nucleic acid sequence selected from the group consisting of: SEQ ID NO:19, SEQ ID NO:20, SEQ ID NO:21 and complementary sequences thereof.

Interestingly, homology was also identified in an element present in several mRNA bound by HuR (FIG. 5). Underligned Is the core of the consensus. It is proposed herein that this element constitutes a new HuR binding consensus sequence. Since this is the element of androgen receptor mRNA 3′UTR bound both by HuR and αCP, it could also represent a consensus for protein binding when both HuR and αCP are present on the RNA. Therefore, another aspect of the invention concerns an isolated or purified nucleic acid molecule comprising a mRNA stabilizing element for HuR binding, this stabilizing element comprising a nucleic acid sequence as set forth in SEQ ID NO:22, SEQ SEQ ID NO:23, and complementary sequences thereof.

Other aspects of the present invention turn to profit the knowledge of the above-described mRNA stabilizing elements and consensus sequences. More particularly, the invention also concerns a method for modulating gene expression in a cell, comprising modulating binding of a αCP polypeptide to a mRNA stabilizing element of a nucleic acid molecule that is present into the cell, the mRNA stabilizing element comprising SEQ ID NO: 19, SEQ ID NO: 20 and/or SEQ ID NO:21. The invention also concerns a method for modulating gene expression in a cell, comprising modulating binding of a HuR polypeptide to a mRNA stabilizing element of a nucleic acid molecule that is present into the cell, the mRNA stabilizing element comprising SEQ ID NO: 22, and/or SEQ ID NO:23.

Preferably, the nucleic acid molecule consists of a mRNA and the binding of the αCP polypeptide or the HuR polypeptide to the mRNA stabilizing element(s) increases the stability of the mRNA and/or its resistance to degradation. In an embodiment, the binding to the sequence(s) is increased, thereby increasing the stability of the mRNA(s) and the expression of the gene. In another embodiment, the binding to the sequence is inhibited or blocked, thereby reducing the stability of the mRNA(s) and the expression of the gene. A non limitative list of genes for which expression could be modulated according to the invention includes: those encoding Vascular Endothelial Growth Factor (VEGF), α-globin, Erythropoietin (EPO), 15-Lipoxygenase (15-LOX), Androgen receptor (AR), and p21.

The above-described mRNA stabilizing elements and consensus sequences may also be used for other purposes including but not limited to:

the identification of other genes regulated by similar or identical stabilizing elements;

the design of proteins, peptides or chemicals targeting these mRNA stabilizing elements;

the identification and purification of other binding proteins yet to be discovered or unknown to have a binding affinity to mRNA stabilizing element(s); and

their use as a regulatory element in gene expression.

Therefore, another aspect of the invention concern methods and tools for the identification and/or purification of compounds (proteins, polypeptide, chemicals, etc) having a binding affinity a mRNA stabilizing element as given previously. In a preferred embodiment, nucleic acid molecules comprising the mRNA stabilizing element(s) is bound to a column and theses nucleic acids are used to purify interacting compounds from a pool of compounds (a chemical bank or cell protein extracts for example). This may be achieved by putting in contact the compounds and the nucleic acid molecules of the column, followed by washing all the non-specific interaction. Purification and identification of interacting compound can be done by usual methods (RMN, sequencing).

Also, another very interesting aspect of the invention concerns the use of mRNA stabilizing elements as regulatory elements in gene expression. For instance, the mRNA stabilizing element(s) could be part of artificial expression vectors for an hypoxia-regulated expression at the mRNA level, similar as the hypoxia regulated gene therapy vector described in U.S. Pat. No. 5,681,706 (incorporated herein by reference). The mRNA regulation could consists of a 2^{nd} level of regulation or be used instead of commonly used tissue-specific regulation (1^{st} level).

Therefore, the present invention encompasses an expression vector comprising a mRNA stabilizing element inserted a 3′UTR region of a sequence encoding a gene product. The invention also encompasses a method for expressing a gene product using such vector, and more particularly for an hypoxia-regulated expression cassette (stimulated under conditions of hypoxia). For expressing a gene product under conditions of hypoxia, a vector as defined previously would be introducing into a suitable host cell.

Gene product for which expression could be regulated includes but is not limited to hypoxia inducible factors (HIF), Vascular Endothelial Growth Factors (VEGF), Fibroblast Growth Factors (FGF), a natriuretic pepdites (e.g. ANP, BNP and CNP) and a Developmentally Regulated Endothelial Locus proteins.

### viii) Modulation of Binding Interactions Involving αCP and HuR

Modulation of αCP-HuR binding interaction and interaction of αCP and/or HuR to different mRNAs constitute the core of the present invention. Of course many suitable approaches may be used to modulate such binding.

A binding interaction may be permitted, increased, or stimulated by increasing access or intracellular levels of αCP and/or HuR. In one embodiment, a nucleic acid sequence encoding a polypeptide having the biological activity of a human αCP polypeptide is introduced and expressed in the cell. In another embodiment, a nucleic acid sequence encoding a polypeptide having the biological activity of a human HυP polypeptide is introduced and expressed in the cell. In yet another embodiment, the cell is contacted with a compound selected from the group consisting of a αCP polypeptide, a αCP nucleic acid, a HuR polypeptide, a HuR nucleic acid, and/or drugs which are capable of enhancing αCP and/or HuR expression. In yet another embodiment, the cell is contacted with a drug or chemical increasing the binding affinity of αCP for HuR (and vice versa). In another embodiment, the binding interaction is permitted or increased by using means capable of stimulating or forcing the αCP-HuR, αCP-mRNA and/or HuR-mRNA binding interaction (e.g. fusions proteins having both αCP and HuR binding domains.

The αCP-HuR, αCP-mRNA and/or HuR-mRNA binding interaction may be reduced or blocked by reducing intracellular levels of each or both αCP and HuR polypeptides. In a preferred embodiment, αCP and/or HuR antisense molecules are introduced or expressed into the cell. This can be achieved by intravenous injection, intratissular injection or other local drug delivery using currently available methods. In another embodiment, ribozymes targeting αCP and/or HuR RNA are used. Other means (e.g. antibodies, small molecules, etc) capable of blocking or inhibiting the αCP-HuR, αCP-mRNA and/or HuR-mRNA binding interaction are also suitable. It is also conceivable to block the αCP-mRNA and/or HuR-mRNA binding interaction by targeting specific consensus binding sequence, and more particularly those comprising: SEQ ID NO:19 to 23.

### ix) Upregulation of VEGF Expression for Promoting Angiogenesis

Knowledge of a binding interaction between i) αCP and HuR, and ii) αCP and VEGF mRNA, provides new means for upregulating VEGF and thereby promote angiogenesis in ischemic and non-ischemic tissue of mammals, preferably animal models and humans.

The inventor proposes a model wherein αCP and HuR would both cooperate for stabilization of VEGF mRNA. Indeed, αCP1 and αCP2 are both shown to interact with Poly(A)-Binding Protein (PABP). PABP binds poly(A) tails of mRNA conferring a higher stability to the mRNA. Thus, according to the model: 1) HuR binds to VEGF mRNA; 2) αCP1 interacts with HuR (which probably also brings αCP2 in the complex); 3) it is possible that αCP1 also binds other domains of VEGF mRNA, thus, HuR and αCP binding to the RNA might be done in a cooperative manner; and 4) PABP is recruited by αCP, thus binding with more efficacy to the mRNA poly(A). The end result is a complex binding several parts of the mRNA, including the poly(A), making it more stable and less accessible to degradation (through specific endonucleases that cleaves within HuR binding site, for example). Since HuR itself interacts with the poly(A), it adds another level of stability to the complex.

Therefore, another aspect of the invention concerns methods for modulating VEGF expression in a mammalian cell, the method comprising the step of modulating in the cell a binding interaction between a αCP polypeptide and a HuR polypeptide. In one embodiment, VEGF expression is reduced by inhibiting or blocking the αCP-HuR binding interaction. In another embodiment, VEGF expression is increased by permitting or stimulating the αCP-HuR binding interaction.

Another aspect of the invention concerns methods for increasing VEGF expression in a mammalian cell, the method comprising the step of permitting or stimulating in the cell a binding interaction between a αCP polypeptide and a HuR polypeptide. Preferably, the binding interaction is increased or permitted by increasing intracellular levels of the αCP polypeptide and/or intracellular levels of the HuR polypeptide. Preferably also, the cell consists of a muscular cell located in muscular tissue of a living mammal, and the increased expression of VEGF induces angiogenesis in the muscular tissue of the mammal.

According to another related aspect, the invention concerns a method for modulating VEGF expression in a mammalian cell, the method comprising the step of modulating in the cell a binding interaction between a αCP polypeptide and a VEGF mRNA. In one embodiment, the VEGF expression is reduced by inhibiting or blocking the αCP-VEGF mRNA binding interaction. In another embodiment, VEGF expression is increased by permitting or stimulating the αCP-VEGF mRNA binding interaction.

The invention also provides a method for modulating VEGF mRNA stability or resistance to degradation in a mammalian cell, by modulating in the cell a binding interaction between a αCP polypeptide and the VEGF mRNA. In one embodiment the αCP-VEGF mRNA binding interaction is reduced or blocked, thereby reducing the stability or resistance to degradation of VEGF mRNA. In another embodiment, the αCP-VEGF mRNA binding interaction is permitted or increased, thereby increasing the stability or resistance to degradation of VEGF mRNA. According to the proposed model hereinbefore, binding of αCP to the VEGF mRNA occurs through the intermediary of a HuR polypeptide.

In yet another more specific aspect, the invention also relates to a method for inducing angiogenesis in a mammalian tissue having a plurality of cells. The method comprises the step of permitting or increasing in cells of this tissue a binding interaction between a αCP polypeptide and a VEGF mRNA; and/or a binding interaction between a αCP polypeptide and a HuR polypeptide. Preferably the cells consist of HEK293 cells, Hep3B cells, mammalian skeletal muscular cells, cardiac cells (e.g. cardiomyocytes), bone marrow cells, stroma cells, fibroblasts, smooth muscle cells, skeletal muscle cells, endothelial cells, endothelial progenitor cells or embryonic stem cells or other stem cells.

One of the many different approaches described hereinbefore may be used for blocking or reducing αCP-HuR, αCP-VEGF mRNA and HuR-VEGF mRNA binding interaction.

As mentioned previously in the Background section, stimulation of angiogenesis may be beneficial for the treatment of coronary heart diseases. Delivery of an angiogenic molecule can lead to a higher density of blood vessel in a defined area, giving rise to an increase blood perfusion in said area. Therapeutic angiogenesis treatments can relieve ischemia in coronary artery disease of peripheric artery disease. Angiogenesis promoting proteins can be delivered through direct intramuscular or intramyocardic injections, transcatheter endomyocardial injections or intra-arterial (or intravenous) infusions of the protein. Angiogenic genes can also be delivered through gene transfer using viral (adenovirus, retrovirus, lentivirus, adeno associated virus) based vectors or non-viral vectors (plasmid DNA with or without lipofection, electroporation, protein based transmembrane DNA transfer). These genes can be delivered through an alternative route of administration or through cell based gene delivery. Cells can be modified ex vivo and then administered to the patient by direct intramuscular or intramyocardic injection, transcatheter endomyocardic injection, with or without in vivo electroporation or intraarterial or intravenous infusion of vectors.

Therefore, the human αCP1A and αCP1B sequences of the invention described previously could be advantageously used for angiogenesis purposes. In one embodiment, the cell consists of a cardiac cell located in the heart of a living mammal, and the a αCP and/or HuR nucleic acid sequence is introduced in a plurality of these cardiac cells such that expression of the αCP and/or HuR polypeptides increases VEGF expression and induces angiogenesis in cardiac tissue of the mammal. Introduction of the αCP and/or HuR nucleic acid sequences may be done by using a vector as defined previously or by any suitable technique known in the art.

In another embodiment, the cell consists of an isolated muscular cell (preferably skeletal muscular cell), and this cell is genetically modified so as to express the αCP and/or HuR polypeptide. Genetically modified αCP and/or HuR expressing cells are then transplanted in the tissue (e.g. cardiac tissue) of a compatible mammalian recipient. More preferably, the transplantation is autologous (the cells are isolated from muscular tissue, such as leg, of the recipient), and the cells are transplanted to the recipient (such as an injection in the scar of the heart) in an amount that is sufficient to induce angiogenesis locally or in surrounding transplanted tissue.

In yet another embodiment, the cell consists of a muscular cell located in the muscular tissue of a living mammal, and expression of the αCP and/or HuR polypeptide induces angiogenesis in the muscular tissue of the mammal (autologous or heterologous transplantation). This method is particularly useful for treating peripheral artery diseases (e.g. ischemia in the legs due to femoral or upstream artery obstruction in humans).

The nucleotide sequence may be introduced in the cell or tissue using well known methods. Indeed, the sequence(s) may be introduced directly in the cells of a given tissue, injected in the tissue, or introduced via the transplantation of previously genetically modified compatible cells. Methods for introducing a nucleotide sequence into eukaryote cells such as mammalian muscular cells or for genetically modifying such cells are well known in the art. For instance, this may be achieved with adenoviral vectors, plasmid DNA transfer (naked DNA or complexed with liposomes) or electroporation. If necessary, a person skilled in the art may look at Isner J., (Nature (2002), 415:234-239) for a review of myocardial gene therapy methods and to US patent application US20010041679A1 or U.S. Pat. No. 5,792,453 which provides methods of gene transfer-mediated angiogenesis therapy. Preferably, the level of expression of the transcription factor(s) is such that the angiogenesis-related gene is expressed at a level that is sufficient to induce angiogenesis locally or in surrounding tissue. For better controlling its expression and selectivity, the transcription factor may be inducible.

### x) Downmodulation of αCP-HuR, αCP-mRNA and HuR-mRNA Binding Interaction

As mentioned previously, androgen receptor (AR) and 15-lipoxygenase (15-LOX) seem to be correlated with cancer cell proliferation. Accordingly, downmodulation of AR and/or 15-LOX could be used to prevent and/or treat these tumors. Therefore, another aspect of the invention relates to a method for reducing tumoral cell survival or for eliminating a tumoral cell in a mammal, comprising reducing expression of androgen receptor (AR) and/or 15-lipoxygenase (15-LOX). The method comprises blocking or reducing a binding interaction between at least two compounds selected from the group consisting of:

i) a αCP polypeptide and a HuR polypeptide;

ii) a αCP polypeptide and a VEGF mRNA;

iii) a HuR polypeptide and a VEGF mRNA and

iv) a HuR polypeptide and a 15-LOX mRNA.

One of the many different approaches described hereinbefore may be used for blocking or reducing αCP-HuR, αCP-mRNA and HuR-mRNA binding interaction.

### xi) Upmodulation of αCP-HuR, αCP-mRNA and HuR-mRNA Binding Interaction

It is known that erythropoietin (EPO) is involved in erythropoiesis while α-globin is an important component of the oxygen carrier in erythrocytes. Accordingly, upmodulation of EPO and/or α-globin could be used to reduce anemia. Therefore, another aspect of the invention relates to a method for reducing anemia in a mammal. The method comprises increasing expression of erythropoietin and/or α-globin by permitting or increasing a binding interaction between at least two compounds selected from the group consisting of:

i) a αCP polypeptide and a HuR polypeptide;

ii) a HuR polypeptide and a EPO mRNA;

iii) a HuR polypeptide and a α-globin mRNA.

Similarly, it is known that Tyrosine Hydroxylase (TH) is involved in mammalian diseases involving reduced levels of L-DOPA including dystonia, Parkingson's disease, and mood and major disorders such as bipolar, depressive and schizoaffective disorders. Accordingly, upmodulation of TH could be useful for treating them. Therefore, another aspect of the invention relates to a method for treating a mammalian disease involving reduced levels of L-DOPA, the method comprising increasing Tyrosine Hydroxylase (TH) expression by permitting or increasing a binding interaction between at least two compounds selected from the group consisting of:

i) a αCP polypeptide and a HuR polypeptide;

ii) a HuR polypeptide and a TH mRNA.

One of the many different approaches described hereinbefore may be used for permitting or increasing a αCP-HuR, αCP-mRNA and/or HuR-mRNA binding interaction.

### xii) Administration of Modulators of αCP-HuR, αCP-mRNA and HuR-mRNA Binding Interaction

A αCP polypeptide, HuR polypeptide, antibody or modulator may be administered within a pharmaceutically acceptable diluent, carrier, or excipient, in unit dosage form. Conventional pharmaceutical practice may be used to provide suitable formulations or compositions to administer αCP polypeptide, HuR polypeptide, or modulator thereof to patients. Administration may begin before the patient is symptomatic. Any appropriate route of administration may be employed, for example, administration may be parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a αCP polypeptide, HuR polypeptide or modulatory compound may be combined with more traditional therapies for the disease such as surgery, steroid therapy, or chemotherapy.

According to a preferred embodiment, the αCP polypeptide, HuR polypeptide or modulatory compound would be incorporated in a pharmaceutical composition with a pharmaceutically acceptable carrier. The amount of active compound present in the composition of the present invention is a therapeutically effective amount. A therapeutically effective amount is that amount necessary so that the compound performs its biological function without causing overly negative effects in the host to which the composition is administered. The exact amount of compound to be used and composition to be administered will vary according to factors such as the type of compound, the compound biological activity, the type of condition being treated, the mode of administration, as well as the other ingredients in the composition. For preparing and administering pharmaceutical compositions comprising the same, methods well known in the art may be used.

### xiii) Identification of Molecules that Modulate αCP-HuR, αCP-mRNA and HuR-mRNA Binding Interaction

It is conceivable that small molecule analogs could be used and administered to act as modulator of αCP-HuR, αCP-mRNA and HuR-mRNA binding interaction and in this manner produce a desired physiological effect. Methods for finding such molecules are provided herein.

αCPs cDNA may be used to facilitate the identification of molecules that increase or decrease αCPs expression. In one approach, candidate molecules are added, in varying concentration, to the culture medium of cells expressing αCP1A, αCP1B, αCP2A and/or αCP2B mRNA. αCP expression is then measured (or expression of another gene, such as VEGF which expression is regulated by αCP), for example, by Northern blot analysis using a αCP cDNA, or cDNA or RNA fragment, as a hybridization probe. The level of αCP expression in the presence of the candidate molecule is compared to the level of αCP expression in the absence of the candidate molecule, all other factors (e.g. cell type and culture conditions) being equal.

Compounds that modulate the level of αCP1A, αCP1B, αCP2A and/or αCP2B may be purified, or substantially purified, or may be one component of a mixture of compounds such as an extract or supernatant obtained from cells. In an assay of a mixture of compounds, αCP expression is tested against progressively smaller subsets of the compound pool (e.g., produced by standard purification techniques such as HPLC or FPLC) until a single compound or minimal number of effective compounds is demonstrated to modulate αCP expression.

Compounds may also be screened for their ability to modulate αCP and/or HuR biological activity and more particularly modulating αCP-HuR, αCP-mRNA and/or HuR-mRNA binding interaction. In this approach, the biological activity of αCP and/or HuR, or of a cell expressing αCP and HuR, (e.g. kidney, heart or muscle cell) in the presence of a candidate compound is compared to the biological activity in its absence, under equivalent conditions. Again, the screen may begin with a pool of candidate compounds, from which one or more useful modulator compounds are isolated in a step-wise fashion. The αCP, HuR, or cell biological activity may be measured by any suitable standard assay.

The effect of candidate molecules on αCP- or HuR-biological activity may, instead, be measured at the level of translation by using the general approach described above with standard protein detection techniques, such as Western blotting or immunoprecipitation with a αCP- or HuR-specific antibody (for example, the αCP1A, αCP1B, αCP2A and/or αCP2B antibodies described herein).

Another method for detecting compounds that modulate the activity of αCP or HuR is to screen for compounds that interact physically with αCP and/or HuR. Depending on the nature of the compounds to be tested, the binding interaction may be measured using methods such as enzyme-linked immunosorbent assays (ELISA), filter binding assays, FRET assays, scintillation proximity assays, microscopic visualization, immunostaining of the cells, in situ hybridization, PCR, etc.

According to a more specific aspect, the invention provides a method for selecting a compound that is capable of reducing gene expression. In one embodiment, the method comprises:

contacting a functional αCP polypeptide and a functional HuR polypeptide in the presence of a compound to be tested;

measuring a binding interaction between the functional αCP polypeptide and the functional HuR polypeptide;

whereby a compound is selected when the binding interaction is measurably reduced in presence of the compound.

According to an even more specific aspect, the invention provides a method for selecting a compound that is capable of reducing VEGF expression, the method comprising:

contacting, in presence of a compound to be tested, a functional αCP polypeptide with a nucleic acid molecule comprising a functional mRNA stabilizing element;

measuring binding of the functional αCP polypeptide to the nucleic acid molecule;

whereby a compound is selected when said binding interaction is measurably reduced in presence of the compound.

Preferably, the nucleic acid molecule consists of a RNA. Preferably also, the functional mRNA stabilizing element comprises a sequence selected from the group consisting of: SEQ ID NO: 19 to 23.

In another embodiment, the method for selecting a compound that is capable of reducing gene expression comprises the steps of:

a) providing a test tube comprising: (i) a functional αCP polypeptide, (ii) a functional HuR polypeptide, and (iii) at least one compound to be tested;

b) measuring a binding interaction between the functional αCP polypeptide and the functional HuR polypeptide; and

c) comparing the measure obtained with a control value;

whereby a compound is selected when the binding interaction is measurably reduced as compared to the control value.

The measurement of the binding interaction may be carried out using different methods such as: enzyme-linked immunosorbent assay (ELISA), filter binding assay, FRET assay, scintillation proximity assay, microscopic visualization, immunostaining of cell, in situ hybridization, SDS PAGE electrophoresis, and reporter gene expression (e.g. β-galactosidate, chloramphenicol acetyl transferase, green fluorescent protein (GFP), Blue fluorescent protein).

In yet another embodiment, the method for selecting a compound that is capable of reducing gene expression comprises the steps of:

a) providing a cell expressing: (i) a functional αCP polypeptide, (ii) a functional HuR polypeptide, and (iii) mRNAs encoded by the gene;

b) contacting a potential compound with the cell; and

c) measuring protein expression of the gene protein and/or mRNA degradation of mRNAs produced by the gene;

whereby a compound is selected when measures obtained at (c) are measurably increased as compared to a control value.

Preferably, the gene encodes a protein selected from the group consisting of: Vascular Endothelial Growth Factor (VEGF), α-globin, Erythropoietin (EPO), 15-Lipoxygenase (15-LOX), and Androgen receptor (AR).

A molecule that promotes an increase in αCP or HuR expression or binding activity is considered particularly useful to the invention; such a molecule may be used, for example, as a therapeutic to increase cellular levels of αCP or HuR and thereby exploit the ability of these polypeptides to stabilize mRNAs, increase gene expression and promote and/or induce angiogenesis.

A molecule that decreases αCP or HuR activity (e.g., by decreasing αCP or HuR expression or binding activity) may be used to decrease and/or block angiogenesis and/or cellular proliferation. This would be advantageous in the treatment of cancer as described previously.

The scope of the invention further encompasses molecules that effectively modulate αCP or HuR gene expression or polypeptide activity and which are found by the methods described above. More particularly, the invention encompasses inhibitors of gene expression identifiable via any one of the methods defined previously. Such compounds may be tested further in animal models. If they continue to function successfully in an in vivo setting, they may be used as therapeutics.

### EXAMPLE 1

### αCP1: a Potential Angiogenic Agent

The following example is illustrative of the wide range of applicability of the present invention and is not intended to limit its scope. Modifications and variations can be made therein without departing from the spirit and scope of the invention. Although any method and material similar or equivalent to those described herein can be used in the practice for testing of the present invention, the preferred methods and materials are described.

Introduction.

VEGF mRNA is a labile messenger that is stabilized in hypoxia. That stabilization occurs through a discrete element in the 3′ untranslated region of the messenger. The protein HuR binds to that element as a complex with unknown proteins, in a hypoxia-specific fashion. Since HuR is present in normoxia as well as in hypoxia, these unknown proteins might thus be responsible for the hypoxia regulation of VEGF mRNA stabilization. As such, the genes encoding those proteins could be useful as angiogenic agents.

In the present example, it is shown that αCP1 can efficiently be used to increase VEGF levels. This action is accomplished through stabilization of VEGF mRNA, resulting in accumulation of the mRNA and thus higher levels of the protein. Also, αCP1 could stabilize other angiogenic factors.

The ability of αCP1 to stabilize VEGF mRNA and other hypoxia regulated mRNAs was tested. Full length αCP1 and αCP2 sequences were therefore isolated from 293 cells.

### Experimental Protocol and Results

Bank Construction

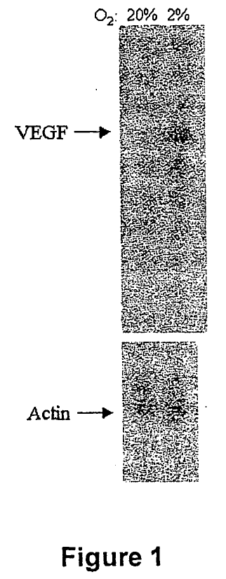

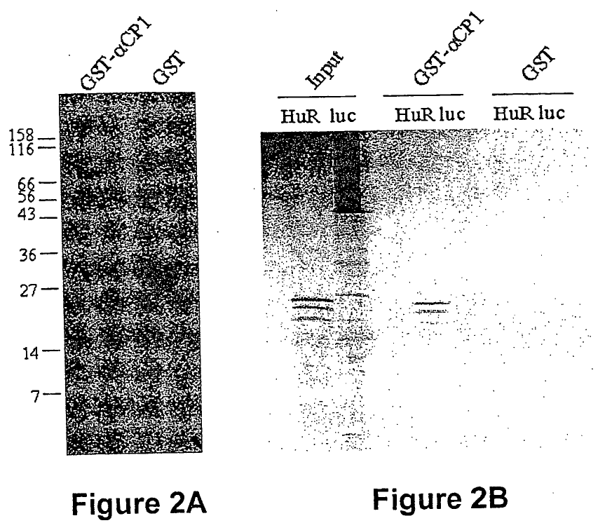

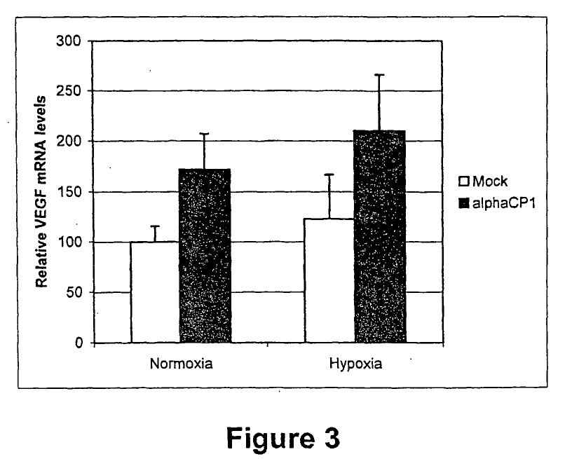

HEK 293 cells were incubated in hypoxia (2% O₂, 5% CO₂) for 24 hours and total RNA was isolated and analyzed on Northern blot with VEGF and actin probes, along with RNA isolated from 293 cells grown in normoxic conditions (FIG. 1). cDNAs derived from hypoxic HIK 293 cells mRNA were inserted in pGAP10™ (ClonTech), transformed into DH10B strain of E. coli.

Yeast Two-Hybrids Screening

HuR partners were isolated using a two-hybrid system (Matchmaker™ Two-Hybrid System 3, ClonTech). That system allows the screening of a cDNA bank to identify clones interacting with a ‘bait’ protein. HuR sequence was used as a bait to screen the hypoxic HEK 293 cDNA bank. Specific interaction of a clone of the bank with the bait HuR allowed expression of selective genes and isolation of these clones. Positive clones were purified and sequenced and several clones contained an insert encoding αCP1.

Full Length αCP Sequences

Full length αCP1 and αCP2 cDNA were amplified by RT-PCR (Marathon™ cDNA Amplification Kit, ClonTech) with human heart RNA (ClonTech) as template and oligonucleotides GAC CCT GCG ACT ACG CTG CGG ACT C (SEQ ID NO:11) and GGA TCC CAG CAT TAA CAG CTG AAC AG (SEQ ID NO:12) as primers for αCP1 and GCT CCC CAG MC ACT GCT CGA CAT G (SEQ ID NO:13) and CCT GGA ATC GCT GAC TGT TCA GM AC (SEQ ID NO:14) for αCP2. The amplification product was inserted in pBluescript™ and sequenced, leading to the identification of αCP1A, αCP1B. αCP2A and αCP2B.

GST Pull Down Assay

To prepare a GST-αCP1 fusion, the αCP1A cDNA was inserted in pGEX-2T™ (Amersham). GST-αCP1A and GST beads were prepared (Gluthatione Sepharose™ 4B, Amersham) according to instructions from the manufacturer and an aliquot was assayed on SDS-PAGE revealed with GelCode™ Blue (Pierce). Radiolabeled HuR and luciferase were prepared by in vitro transcription-translation (TNT Coupled Reticulocyte Lysate System™, Promega) using pcDNA3-HuR. Co-purification of HuR with GST-aCP1 proves that there is an interaction between the two proteins.

Transfections

Early passage HEK 293 cells (ATCC) were plated at 1.3×10⁶ cells per plate (60 mm) and grown overnight. 4 μg of sterile plasmid DNA (αCP1A/pcDNA3 expression vector or emply pcDNA3 plasmid) was transfected (PolyFect™ Transfection Reagent, Qiagen) according to the manufacturer's instructions. After a 5 hours transfection, cells were incubated either in normoxia (5% CO₂ normal air at 37° C.) or in hypoxia (5% CO₂, 2% O₂ at 37° C.) for 24 hours (n=5 for each transfections in each conditions). Total RNA was isolated by (Rneasy™ Mini Kit, Qiagen) extractions and treated with DNAse I (DNA-free™, Ambion).

VEGF Quantification

Messenger RNAs were reverse transcribed with random primers (RetroScript™, Ambion). VEGF165 and actin were amplified from these samples by radioactive PCR, using the oligos CCCTGATGAGATCGAGTACATCIT (SEQ ID NO:15) and AGCAAGGCCCACAGGGATTI (SEQ ID NO:16), CACAGGCATTGTGATGGACTC (SEQ ID NO:17) and GCTCAGGAGGAGCAATGATCT (SEQ ID NO:18) respectively. The number of amplification cycles was carefully kept within the linear range of the PCR for both genes. Intensity of each band was measured and relative expression was calculated as VEGF intensity/actin intensity. Results are expressed as mean±SED. This result shows that a higher level of αCP1 protein can lead to a higher level of VEGF mRNA. This is probably the result in the stabilization of VEGF mRNA by αCP1, which might be a rate limiting component of the VEGF mRNA stability cascade.

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and failing within the scope of the invention or the limits of the appended claims.

1. A method for modulating gene expression in a cell, comprising modulating a binding interaction between a αCP polypeptide and a HuR polypeptide.

2. The method of claim 1, wherein said gene consists of a gene which gives rise to mRNAs on which said αCP polypeptide and/or said HuR polypeptide binds.

3. The method of claim 2, wherein said binding increases stability of said mRNA(s), thereby increasing expression of said gene.

4. The method of claim 1, comprising reducing expression of said gene by inhibiting or blocking said binding interaction.

5. The method of claim 1, comprising increasing expression of said gene by permitting or increasing said binding interaction.

6. The method of claim 1, wherein said gene encodes a protein selected from the group consisting of: Vascular Endothelial Growth Factor (VEGF), α-globin, Erythropoietin (EPO), 15-Lipoxygenase (15-LOX), and Androgen receptor (AR).

7. A method for modulating VEGF expression in a mammalian cell, the method comprising the step of modulating in said cell a binding interaction between a αCP polypeptide and a HuR polypeptide.

8. The method of claim 7, wherein said VEGF expression is reduced by inhibiting or blocking said binding interaction.

9. A method for increasing VEGF expression in a mammalian cell, the method comprising the step of permitting or stimulating in said cell a binding interaction between a αCP polypeptide and a HuR polypeptide.

10. The method of claim 9, wherein said binding interaction is increased or permitted by increasing intracellular levels of said αCP polypeptide and/or intracellular levels of said HuR polypeptide.

11. The method of claim 7, wherein said cell consists of a muscular cell located in the muscular tissue of a living mammal, and wherein expression of said increased VEGF expression induces angiogenesis in the muscular tissue of said mammal.

12. A method for modulating VEGF expression in a mammalian cell, the method comprising the step of modulating in said cell a binding interaction between a αCP polypeptide and a VEGF mRNA.

13. The method of claim 12, wherein said VEGF expression is reduced by inhibiting or blocking said binding interaction.

14. The method of claim 12, wherein said VEGF expression is increased by permitting or stimulating said binding interaction.

15. A method for modulating VEGF mRNA stability or resistance to degradation in a mammalian cell, the method comprising the step of modulating in said cell a binding interaction between a αCP polypeptide and said VEGF mRNA.

16. The method of claim 15, wherein said binding interaction is reduced or blocked, thereby reducing the stability or resistance to degradation of the VEGF mRNA.

17. The method of claim 15, wherein said binding interaction is permitted or increased, thereby increasing the stability or resistance to degradation of the VEGF mRNA.

18. The method of claim 15, wherein binding of said αCP polypeptide to VEGF mRNA occurs through the intermediary of a HuR polypeptide.

19. A method for inducing angiogenesis in a mammalian tissue having a plurality of cells, the method comprising the step of permitting or increasing in cells of said tissue a binding interaction:
between a αCP polypeptide and a VEGF mRNA; and/or
between a αCP polypeptide and a HuR polypeptide.

20. The method of claim 19, wherein said binding interaction is permitted or increased by a method selected from the group consisting of:
introducing and expressing in at least some of said cells a nucleic acid sequence encoding a polypeptide having the biological activity of a human αCP polypeptide;
introducing and expressing in at least some of said cells a nucleic acid sequence encoding a polypeptide having the biological activity of a human a HuR polypeptide;
contacting said tissue with a compound selected from the group consisting of a αCP polypeptide, a αCP nucleic acid, a HuR polypeptide, a HuR nucleic acid, and drugs capable of enhancing αCP and/or HuR expression.

21. The method of claim 19, wherein said cells are selected from the group consisting of HEK293 cells, Hep3B cells, mammalian skeletal muscular cells, cardiac cells, bone marrow cells, fibroblasts, smooth muscle cells, endothelial cells, endothelial progenitor cells and embryonic stem cells.

22. A method for modulating gene expression in a cell, comprising modulating binding of αCP polypeptide to a mRNA stabilizing element of a nucleic acid molecule that is present into said cell, said mRNA stabilizing element comprising SEQ ID NO:19, SEQ ID NO:20 and/or SEQ ID NO: 21.

23. The method of claim 22, wherein said nucleic acid molecule consists of a mRNA and wherein the binding of the αCP polypeptide to the mRNA stabilizing element increases the stability and/or resistance to degradation of said mRNA.

24. The method of claim 23, wherein said binding is increased, thereby increasing the stability of the nucleic acid molecule and the expression of the gene.

25. The method of claim 1, wherein said binding is inhibited or blocked, thereby reducing the stability of the nucleic acid molecule and the expression of the gene.

26. The method of claim 22, wherein said gene encodes a protein selected from the group consisting of: Vascular Endothelial Growth Factor (VEGF), α-globin, Erythropoietin (EPO), 15-Lipoxygenase (15-LOX), and Androgen receptor (AR)

27. An isolated or purified nucleic acid molecule comprising a mRNA stabilizing element, said mRNA stabilizing element comprising a nucleic acid sequence selected from the group consisting of: SEQ ID NO:19, SEQ ID NO:20, SEQ ID NO:21, SEQ ID NO:22, SEQ ID NO:23, and complementary sequences thereof.

28. An expression vector comprising a nucleic acid sequence encoding a gene product, said vector further comprising a nucleic acid molecule as defined in claim 27 in a 3′UTR region of the sequence encoding said gene product.

29. A method for expressing a gene product under conditions of hypoxia, comprising introducing into a host cell a vector as defined in claim 28.

30. The method of claim 29, wherein said gene product is selected from the group consisting of: a hypoxia inducible factor (HIF), a Vascular Endothelial Growth Factor (VEGF), a Fibroblast Growth Factor (FGF), a natriuretic pepdite and a Developmentally Regulated Endothelial Locus protein.

31. A method for selecting a compound that is capable of reducing gene expression, the method comprising:
contacting a functional αCP polypeptide and a functional HuR polypeptide in the presence of a compound to be tested;
measuring a binding interaction between said functional αCP polypeptide and said functional HuR polypeptide;
whereby a compound is selected when said binding interaction is measurably reduced in presence of the compound.

32. A method for selecting a compound that is capable of reducing VEGF expression, the method comprising:
contacting, in presence of a compound to be tested, a functional αCP polypeptide with a nucleic acid molecule comprising a functional mRNA stabilizing element;
measuring binding of said functional αCP polypeptide to said nucleic acid molecule;
whereby a compound is selected when said binding interaction is measurably reduced in presence of the compound.

33. The method of claim 32, wherein said nucleic acid molecule consists of a RNA.

34. The method of claim 33, wherein RNA consists of a VEGF mRNA or a fragment thereof.

35. The method claim 32, wherein said functional mRNA stabilizing element comprises a sequence that is selected from the group consisting of: SEQ ID NO:19, SEQ ID NO:20, SEQ ID NO:21, SEQ ID NO:22, and SEQ ID NO:23.

36. An in vitro method for selecting a compound that is capable of reducing gene expression, the method comprising:
a) providing a test tube comprising: (i) a functional αCP polypeptide, (ii) a functional HuR polypeptide, and (iii) at least one compound to be tested;
b) measuring a binding interaction between said functional αCP polypeptide and said functional HuR polypeptide; and
c) comparing said measure with a control value;
whereby a compound is selected when said binding interaction is measurably reduced as compared to the control value.

37. The method claim 31, wherein measurement of said binding interaction comprises carrying out a method selected from the group consisting of: enzyme-linked immunosorbent assay (ELISA), filter binding assay, FRET assay, scintillation proximity assay, microscopic visualization, immunostaining of cell, in situ hybridization, SDS PAGE electrophoresis, and reporter gene expression.

38. A method for selecting a compound that is capable of reducing gene expression, the method comprising:
a) providing a cell expressing: (i) a functional αCP polypeptide, (ii) a functional HuR polypeptide, and (iii) mRNAs encoded by a gene for which a reduced expression is desired;
b) contacting a potential compound with the cell; and
c) measuring protein expression of said gene protein and/or mRNA degradation of mRNAs produced by said gene;
whereby a compound is selected when measures obtained at (c) are measurably increased as compared to a control value.

39. The method of claim 31, wherein said gene encodes a protein selected from the group consisting of: Vascular Endothelial Growth Factor (VEGF), α-globin, Erythropoietin (EPO), 15-Lipoxygenase (15-LOX), and Androgen receptor (AR).

40. An inhibitor of gene expression identifiable via the methods of claim 31.

41. A method for reducing tumoral cell survival or for eliminating a tumoral cell in a mammal, comprising reducing expression of androgen receptor (AR), Vascular Endothelial Growth Factor VEGF and/or 15-lipoxygenase (15-LOX) by blocking or reducing a binding interaction between at least two compounds selected from the group consisting of:
i) a αCP polypeptide and a HuR polypeptide;
ii) a αCP polypeptide and a VEGF mRNA;
iii) a HuR polypeptide and a 15-LOX mRNA.

42. A method for reducing anemia in a mammal, the method comprising increasing expression of erythropoietin and/or α-globin by permitting or increasing a binding interaction between at least two compounds selected from the group consisting of:
i) a αCP polypeptide and a HuR polypeptide;
ii) a HuR polypeptide and a EPO mRNA;
iii) a HuR polypeptide and a α-globin mRNA.

43. A method for treating a mammalian disease involving reduced levels of L-DOPA, the method comprising increasing Tyrosine Hydroxylase (TH) expression by permitting or increasing a binding interaction between at least two compounds selected from the group consisting of:
a) a αCP polypeptide and a HuR polypeptide;
b) a HuR polypeptide and a TH mRNA.

44. An isolated or purified nucleic acid molecule comprising a sequence selected from the group consisting of:
a) a sequence as set forth in SEQ ID NO: 1 or 3;
b) a sequence encoding an amino acid sequence as set forth in SEQ ID NO: 2 or 4;
c) a sequence as set forth in SEQ ID NO: 5 or 7; and
d) a sequence encoding an amino acid sequence as set forth in SEQ ID NO: 6 or 8.

45. An isolated or purified protein comprising an amino acid sequence selected from the group consisting of:
a) sequences encoded by a nucleic acid of claim 44;
b) sequences comprising an amino acid sequence as set forth in SEQ ID NO: 2, SEQ ID NO: 4, SEQ ID NO: 6 or SEQ ID NO:8.

46. A cloning or expression vector comprising the nucleic acid molecule of claim 44.

47. A transformed or transfected cell that contains the nucleic acid molecule of claim 44.

48. A transgenic animal generated from the cell of claim 47, wherein said nucleic acid molecule is expressed in said transgenic animal.

49. An isolated or purified antibody that specifically binds to an isolated or purified protein as defined in claim 45.

50. A method for determining the amount of a human αCP polypeptide in a biological sample, comprising the step of contacting said sample with the antibody of claim 49.

51. A kit for determining the amount of a human αCP polypeptide in a biological sample, said kit comprising the antibody of claim 49, and at least one element selected from the group consisting of instructions for using said kit, reaction buffer(s), and enzyme(s).

52. A method for producing a human αCP polypeptide comprising:
providing a cell transformed with a nucleic acid sequence according to claim 1 positioned for expression in said cell;
culturing said transformed cell under conditions suitable for expressing said nucleic acid; and
producing said human αCP polypeptide.

53. A transformed or transfected cell that contains the vector of claim 46.


SEQUENCE LISTING (24 sequences)

SEQ ID NO: 1 - DNA, 1417 bp, Homo sapiens
  CDS (168)..(1238)
gaccctgcgactacgctgcggactcccgcccgctcccgctcgctcccgcggccctcgctcgcctcgcgccggcagttttgggcctacacctcccctccccccgccagccgccaaagacttgaccacgtaacgagcccaactcccccgaacgccgcccgccgctcgccatggatgccggtgtgactgaaagtggactaaatgtgactctcaccattcggcttcttatgcacggaaaggaagtaggaagcatcattgggaagaaaggggagtcggttaagaggatccgcgaggagagtggcgcgcggatcaacatctcggaggggaattgtccggagagaatcatcactctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtacgggggcgcaggtccaggtggcggtggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctactcgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatctgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctagaacagtgtaggttccctcaataacccctttctgctgttctcccatgatccaactgtgtaatttctggtcagtgattccaggttttaaataatttgtaagtgttcagtttctacacaactttatcatccgctaagaatttaaaaatcacattctctgttcagctgttaatgctgggatcc

SEQ ID NO: 2 - protein, 356 aa, Homo sapiens
MDAGVTESGLNVTLTIRLLMHGKEVGSIIGKKGESVKRIREESGARINISEGNCPERIITLTGPTNAIFKAFAMIIDKLEEDINSSMTNSTAASRPPVTLRLVVPATQCGSLIGKGGCKIKEIRESTGAQVQVAVDMLPNSTERAITIAGVPQSVTECVKQICLVMLETLSQSPQGRVMTIPYQPMPASSPVICAGGQDRCSDAAGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEICQMSGAQIKIANPVEGSSGRQVTITGSAASISLAQYLINARLSSEKGMGCS

SEQ ID NO: 3 - DNA, 1384 bp, Homo sapiens
  CDS (168)..(1205)
gaccctgcgactacgctgcggactcccgcccgctcccgctcgctcccgcggccctcgctcgcctcgcgccggcagttttgggcctacacctcccctccccccgccagccgccaaagacttgaccacgtaacgagcccaactcccccgaacgccgcccgccgctcgccatggatgccggtgtgactgaaagtggactaaatgtgactctcaccattcggcttcttatgcacggaaaggaagtaggaagcatcattgggaagaaaggggagtcggttaacatctcggaggggaattgtccggagagaatcatcactctgaccggccccaccaatgccatctttaaggctttcgctatgatcatcgacaagctggaggaagatatcaacagctccatgaccaacagtaccgcggccagcaggcccccggtcaccctgaggctggtggtgccggccacccagtgcggctccctgattgggaaaggcgggtgtaagatcaaagagatccgcgagagtacgggggcgcaggtccaggtggcgggggatatgctgcccaactccaccgagcgggccatcaccatcgctggcgtgccgcagtctgtcaccgagtgtgtcaagcagatttgcctggtcatgctggagacgctctcccagtctccgcaagggagagtcatgaccattccgtaccagcccatgccggccagctccccagtcatctgcgcgggcggccaagatcggtgcagcgacgctgcgggctacccccatgccacccatgacctggagggaccacctctagatgcctactcgattcaaggacaacacaccatttctccgctcgatctggccaagctgaaccaggtggcaagacaacagtctcactttgccatgatgcacggcgggaccggattcgccggaattgactccagctctccagaggtgaaaggctattgggcaagtttggatgcatctactcaaaccacccatgaactcaccattccaaataacttaattggctgcataatcgggcgccaaggcgccaacattaatgagatccgccagatgtccggggcccagatcaaaattgccaacccagtggaaggctcctctggtaggcaggttactatcactggctctgctgccagtattagtctggcccagtatctaatcaatgccaggctttcctctgagaagggcatggggtgcagctagaacagtgtaggttccctcaataacccctttctgctgttctcccatgatccaactgtgtaatttctggtcagtgattccaggttttaaataatttgtaagtgttcagtttctacacaactttatcatccgctaagaatttaaaaatcacattctctgttcagctgttaatgctgggatcc

SEQ ID NO: 4 - protein, 345 aa, Homo sapiens
MDAGVTESGLNVTLTIRLLMHGKEVGSIIGKKGESVNISEGNCPERIITLTGPTNAIFKAFAMIIDKLEEDINSSMTNSTAASRPPVTLRLVVPATQCGSLIGKGGCKIKEIRESTGAQVQVAGDMLPNSTERAITIAGVPQSVTECVKQICLVMLETLSQSPQGRVMTIPYQPMPASSPVICAGGQDRCSDAAGYPHATHDLEGPPLDAYSIQGQHTISPLDLAKLNQVARQQSHFAMMHGGTGFAGIDSSSPEVKGYWASLDASTQTTHELTIPNNLIGCIIGRQGANINEIRQMSGAQIKIANPVEGSSGRQVTITGSAASISLAQYLINARLSSEKGMGCS

SEQ ID NO: 5 - DNA, 1162 bp, Homo sapiens
  CDS (23)..(1072)
gctccccagaacactgctcgacatggacaccggtgtgattgaaggtggattaaatgtcactctcaccatccggctacttatgcatggaaaggaagttggcagtatcatcggaaagaaaggagaatcagttaagaagatgcgcgaggagagtggtgcacgtatcaacatctcagaagggaattgtcctgagagaattatcactttggctggacccactaatgccatcttcaaagcctttgctatgatcattgacaaactggaagaggacataagcagctctatgaccaatagcacagctgccagtagacccccggtcaccctgaggctggtggtccctgctagtcagtgtggctctctcattggaaaaggtggatgcaagatcaaggaaatacgagagagtacaggggctcaggtccaggtggcaggggatatgctacccaactcaactgagcgggccatcactattgctggcattccacaatccatcattgagtgtgtcaaacagatctgcgtggtcatgttggagtcccccccgaagggcgtgaccatcccgtaccggcccaagccgtccagctctccggtcatctttgcaggtggtcaggacaggtacagcacaggcagcgacagtgcgagctttccccacaccaccccgtccatgtgcctcaaccctgacctggagggaccacctctagaggcctataccattcaaggacagtatgccattccacagccagatttgaccaagctgcaccagttggcaatgcaacagtctcattttcccatgacgcatggcaacaccggattcagtgcaggtttggatgcatctgctcagactacttctcatgaactcaccattccaaacgatttgattggctgcataatcgggcgtcaaggcgccaaaatcaatgagatccgtcagatgtctggggcgcagatcaaaattgcgaacccagtggaaggatctactgataggcaggttaccatcactggatctgctgccagcattagcctggctcaatatctaatcaatgtcaggctttcctcggagacgggtggcatggggagcagctagaacaatgcagattcatccataatccctttctgctgttcaccaccacccatgatccatctgtgtagtttctgaacagtcagcgattccagg

SEQ ID NO: 6 - protein, 349 aa, Homo sapiens
MDTGVIEGGLNVTLTIRLLMHGKEVGSIIGKKGESVKKMREESGARINISEGNCPERIITLAGPTNAIFKAFAMIIDKLEEDISSSMTNSTAASRPPVTLRLVVPASQCGSLIGKGGCKIKEIRESTGAQVQVAGDMLPNSTERAITIAGIPQSIIECVKQICVVMLESPPKGVTIPYRPKPSSSPVIFAGGQDRYSTGSDSASFPHTTPSMCLNPDLEGPPLEAYTIQGQYAIPQPDLTKLHQLAMQQSHFPMTHGNTGFSAGLDASAQTTSHELTIPNDLIGCIIGRQGAKINEIRQMSGAQIKIANPVEGSTDRQVTITGSAASISLAQYLINVRLSSETGGMGSS

SEQ ID NO: 7 - DNA, 1080 bp, Homo sapiens
  CDS (23)..(991)
gctccccagaacactgctcgacatggacaccggtgtgattgaaggtggattaaatgtcactctcaccatccggctacttatgcatggaaaggaagttggcagtatcatcggaaagaaaggagaatcagttaagaagatgcgcgaggagagtggtgcacgtatcaacatctcagaagggaattgtcctgagagaattatcactttggctggacccactaatgccatcttcaaagcctttgctatgatcattgacaaactggaagaggacataagcagctctatgaccaatagcacagctgccagtagacccccggtcaccctgaggctggtggtccctgctagtcagtgtggctctctcattggaaaaggtggatgcaagatcaaggaaatacgagagagtacaggggctcaggtccaggtggcaggggatatgctacccaactcaactgagcgggccatcactattgctggcattccacaatccatcattgagtgtgtcaaacagatctgcgtggtcatgttggagactctctcccagtcccccccgaagggcgtgaccatcccgtaccggcccaagccgtccagctctccggtcatctttgcaggtggtcaggcctataccattcaaggacagtatgccattccacagccagatttgaccaagctgcaccagttggcaatgcaacagtctcattttcccatgacgcatggcaacaccggattcagtgcaggtttggatgcatctgctcagactacttctcatgaactcaccattccaaacgatttgattggctgcataatcgggcgtcaaggcgccaaaatcaatgagatccgtcagatgtctggggcgcagatcaaaattgcgaacccagtggaaggatctactgataggcaggttaccatcactggatctgctgccagcattagcctggctcaatatctaatcaatgtcaggctttcctcggagacgggtggcatggggagcagctagaacaatgcagattcatccataatcccttttgctgttcaccaccacccatgatccatctgtgtagtttctgaacagtcagcgattccagg

SEQ ID NO: 8 - protein, 322 aa, Homo sapiens
MDTGVIEGGLNVTLTIRLLMHGKEVGSIIGKKGESVKKMREESGARINISEGNCPERIITLAGPTNAIFKAFAMIIDKLEEDISSSMTNSTAASRPPVTLRLVVPASQCGSLIGKGGCKIKEIRESTGAQVQVAGDMLPNSTERAITIAGIPQSIIECVKQICVVMLETLSQSPPKGVTIPYRPKPSSSPVIFAGGQAYTIQGQYAIPQPDLTKLHQLAMQQSHFPMTHGNTGFSAGLDASAQTTSHELTIPNDLIGCIIGRQGAKINEIRQMSGAQIKIANPVEGSTDRQVTITGSAASISLAQYLINVRLSSETGGMGSS

SEQ ID NO: 9 - DNA, 1293 bp, Homo sapiens
  CDS (23)..(1120)
gctccccagaacactgctcgacatggacaccggtgtgattgaaggtggattaaatgtcactctcaccatccggctacttatgcatggaaaggaagttggcagtatcatcggaaagaaaggagaatcagttaagaagatgcgcgaggagagtggtgcacgtatcaacatctcagaagggaattgtcctgagagaattatcactttggctggacccactaatgccatcttcaaagcctttgctatgatcattgacaaactggaagaggacataagcagctctatgaccaatagcacagctgccagtagacccccggtcaccctgaggctggtggtccctgctagtcagtgtggctctctcattggaaaaggtggatgcaagatcaaggaaatacgagagagtacaggggctcaggtccaggtggcaggggatatgctacccaactcaactgagcgggccatcactattgctggcattccacaatccatcattgagtgtgtcaaacagatctgcgtggtcatgttggagactctctcccagtcccccccgaagggcgtgaccatcccgtaccggcccaagccgtccagctctccggtcatctttgcaggtggtcaggacaggtacagcacaggcagcgacagtgcgagctttccccacaccaccccgtccatgtgcctcaaccctgacctggagggaccacctctagaggcctataccattcaaggacagtatgccattccacagccagatttgaccaagctgcaccagttggcaatgcaacagtctcattttcccatgacgcatggcaacaccggattcagtggcattgaatccagctctccagaggtgaaaggctattggggtttggatgcatctgctcagactacttctcatgaactcaccattccaaacgatttgattggctgcataatcgggcgtcaaggcgccaaaatcaatgagatccgtcagatgtctggggcgcagatcaaaattgcgaacccagtggaaggatctactgataggcaggttaccatcactggatctgctgccagcattagcctggctcaatatctaatcaatgtcaggctttcctcggagacgggtggcatggggagcagctagaacaatgcagattcatccataatccctttctgctgttcaccaccacccatgatccatctgtgtagtttctgaacagtcagcgattccaggttttaaatagtttgtaaattttcagtttctacacactttatcatccactcgtgattttttaattaaagcgttttaattccttt

SEQ ID NO: 10 - protein, 365 aa, Homo sapiens
MDTGVIEGGLNVTLTIRLLMHGKEVGSIIGKKGESVKKMREESGARINISEGNCPERIITLAGPTNAIFKAFAMIIDKLEEDISSSMTNSTAASRPPVTLRLVVPASQCGSLIGKGGCKIKEIRESTGAQVQVAGDMLPNSTERAITIAGIPQSIIECVKQICVVMLETLSQSPPKGVTIPYRPKPSSSPVIFAGGQDRYSTGSDSASFPHTTPSMCLNPDLEGPPLEAYTIQGQYAIPQPDLTKLHQLAMQQSHFPMTHGNTGFSGIESSSPEVKGYWGLDASAQTTSHELTIPNDLIGCIIGRQGAKINEIRQMSGAQIKIANPVEGSTDRQVTITGSAASISLAQYLINVRLSSETGGMGSS

SEQ ID NO: 11 - DNA, 25 bp, Artificial sequence
  Synthetic oligonucleotide
gaccctgcgactacgctgcggactc

SEQ ID NO: 12 - DNA, 26 bp, Artificial sequence
  Synthetic oligonucleotide
ggatcccagcattaacagctgaacag

SEQ ID NO: 13 - DNA, 25 bp, Artificial sequence
  Synthetic oligonucleotide
gctccccagaacactgctcgacatg

SEQ ID NO: 14 - DNA, 26 bp, Artificial sequence
  Synthetic oligonucleotide
cctggaatcgctgactgttcagaaac

SEQ ID NO: 15 - DNA, 24 bp, Artificial sequence
  Synthetic oligonucleotide
ccctgatgagatcgagtacatctt

SEQ ID NO: 16 - DNA, 20 bp, Artificial sequence
  Synthetic oligonucleotide
agcaaggcccacagggattt

SEQ ID NO: 17 - DNA, 21 bp, Artificial sequence
  Synthetic oligonucleotide
cacaggcattgtgatggactc

SEQ ID NO: 18 - DNA, 21 bp, Artificial sequence
  Synthetic oligonucleotide
gctcaggaggagcaatgatct

SEQ ID NO: 19 - RNA, 25 nt, Artificial sequence
  Synthetic oligonucleotide
ugcccynccynuccccugnyycncc

SEQ ID NO: 20 - RNA, 7 nt, Artificial sequence
  Synthetic oligonucleotide
ccynccy

SEQ ID NO: 21 - RNA, 10 nt, Artificial sequence
  Synthetic oligonucleotide
uccccugnyy

SEQ ID NO: 22 - RNA, 22 nt, Artificial sequence
  Synthetic oligonucleotide
cuuuuncucucucuuncucucu

SEQ ID NO: 23 - RNA, 13 nt, Artificial sequence
  Synthetic oligonucleotide
cuuuuncucucuc

SEQ ID NO: 24 - RNA, 6 nt, Artificial sequence
  Synthetic oligonucleotide
yycccu